SEQUENCE LISTING (60 sequences)

SEQ ID NO: 1 - DNA, 23 bp, Artificial Sequence
  Synthetic Oligonucleotide
gagagaataagttggggtaaagt

SEQ ID NO: 2 - DNA, 24 bp, Artificial Sequence
  Synthetic Oligonucleotide
taactccatccaaaataacataat

SEQ ID NO: 3 - DNA, 20 bp, Artificial Sequence
  Synthetic Oligonucleotide
ggatgtagggagtttgggtt

SEQ ID NO: 4 - DNA, 23 bp, Artificial Sequence
  Synthetic Oligonucleotide
ttccattccraataaacaataac

SEQ ID NO: 5 - DNA, 24 bp, Artificial Sequence
  Synthetic Oligonucleotide
gatttgtagtgagygttaggagta

SEQ ID NO: 6 - DNA, 24 bp, Artificial Sequence
  Synthetic Oligonucleotide
cctatataactaaacrccaaaacc

SEQ ID NO: 7 - DNA, 22 bp, Artificial Sequence
  Synthetic Oligonucleotide
gttygtgggatttttttagtat

SEQ ID NO: 8 - DNA, 20 bp, Artificial Sequence
  Synthetic Oligonucleotide
ccacttcratccactacaaa

SEQ ID NO: 9 - DNA, 23 bp, Artificial Sequence
  Synthetic Oligonucleotide
ggtttatgtttygggtagtttta

SEQ ID NO: 10 - DNA, 18 bp, Artificial Sequence
  Synthetic Oligonucleotide
ctcctcctcrctctccaa

SEQ ID NO: 11 - DNA, 22 bp, Artificial Sequence
  Synthetic Oligonucleotide
gggtaggttaggattgttagtg

SEQ ID NO: 12 - DNA, 21 bp, Artificial Sequence
  Synthetic Oligonucleotide
ctccracaaattacaaatcac

SEQ ID NO: 13 - DNA, 28 bp, Artificial Sequence
  Synthetic Oligonucleotide
gttattggagtgtttattttgtaggtag

SEQ ID NO: 14 - DNA, 24 bp, Artificial Sequence
  Synthetic Oligonucleotide
caacaccaaaaacatctaaaaacc

SEQ ID NO: 15 - DNA, 25 bp, Artificial Sequence
  Synthetic Oligonucleotide
gtttaygagatttttgagataggaa

SEQ ID NO: 16 - DNA, 25 bp, Artificial Sequence
  Synthetic Oligonucleotide
crcaaccactacttttataacaaac

SEQ ID NO: 17 - DNA, 22 bp, Artificial Sequence
  Synthetic Oligonucleotide
ggggtaggagggttgtagtttg

SEQ ID NO: 18 - DNA, 26 bp, Artificial Sequence
  Synthetic Oligonucleotide
accaacaacaacaaataaaaaatacc

SEQ ID NO: 19 - DNA, 24 bp, Artificial Sequence
  Synthetic Oligonucleotide
ggattggatagtttttgttttgat

SEQ ID NO: 20 - DNA, 26 bp, Artificial Sequence
  Synthetic Oligonucleotide
ctctaactcrttatctacatacctcc

SEQ ID NO: 21 - DNA, 26 bp, Artificial Sequence
  Synthetic Oligonucleotide
gttttttagtttgatattagtagggt

SEQ ID NO: 22 - DNA, 20 bp, Artificial Sequence
  Synthetic Oligonucleotide
tacaacaccaccraacaact

SEQ ID NO: 23 - DNA, 28 bp, Artificial Sequence
  Synthetic Oligonucleotide
tatgattattgtagagtgtttatggaag

SEQ ID NO: 24 - DNA, 24 bp, Artificial Sequence
  Synthetic Oligonucleotide
ctaaacctccacatcctaaaaacc

SEQ ID NO: 25 - DNA, 24 bp, Artificial Sequence
  Synthetic Oligonucleotide
ggttyggagaatgtatttattgag

SEQ ID NO: 26 - DNA, 21 bp, Artificial Sequence
  Synthetic Oligonucleotide
cctccctccaaaactacacct

SEQ ID NO: 27 - DNA, 29 bp, Artificial Sequence
  Synthetic Oligonucleotide
gtggaattaagaaagtttagtagattgtg

SEQ ID NO: 28 - DNA, 21 bp, Artificial Sequence
  Synthetic Oligonucleotide
ctccccatcccaaacacttac

SEQ ID NO: 29 - DNA, 26 bp, Artificial Sequence
  Synthetic Oligonucleotide
taagatattagttgtattttgggttg

SEQ ID NO: 30 - DNA, 23 bp, Artificial Sequence
  Synthetic Oligonucleotide
aatatcaaacctccaaattcaca

SEQ ID NO: 31 - DNA, 23 bp, Artificial Sequence
  Synthetic Oligonucleotide
ggtgcgtttagtggagttttgag

SEQ ID NO: 32 - DNA, 21 bp, Artificial Sequence
  Synthetic Oligonucleotide
ataacracccccaacaaccac

SEQ ID NO: 33 - DNA, 21 bp, Artificial Sequence
  Synthetic Oligonucleotide
tttattatttyggaggtggtg

SEQ ID NO: 34 - DNA, 22 bp, Artificial Sequence
  Synthetic Oligonucleotide
cracttcttactccatctttcc

SEQ ID NO: 35 - DNA, 20 bp, Artificial Sequence
  Synthetic Oligonucleotide
tagttgtgaggaggaggaag

SEQ ID NO: 36 - DNA, 20 bp, Artificial Sequence
  Synthetic Oligonucleotide
cctacctatccraatcactc

SEQ ID NO: 37 - DNA, 21 bp, Artificial Sequence
  Synthetic Oligonucleotide
agatatagacggaagtgggtg

SEQ ID NO: 38 - DNA, 24 bp, Artificial Sequence
  Synthetic Oligonucleotide
aaaaaccaacaactaaaacaatac

SEQ ID NO: 39 - DNA, 26 bp, Artificial Sequence
  Synthetic Oligonucleotide
gtttttatgaaagtttgttggtagag

SEQ ID NO: 40 - DNA, 20 bp, Artificial Sequence
  Synthetic Oligonucleotide
cccactcccactccaaaatc

SEQ ID NO: 41 - DNA, 24 bp, Artificial Sequence
  Synthetic Oligonucleotide
ggattttagtttaggtagatttga

SEQ ID NO: 42 - DNA, 18 bp, Artificial Sequence
  Synthetic Oligonucleotide
tcccaccctcraactcta

SEQ ID NO: 43 - DNA, 19 bp, Artificial Sequence
  Synthetic Oligonucleotide
tggatttgygagaagaaat

SEQ ID NO: 44 - DNA, 24 bp, Artificial Sequence
  Synthetic Oligonucleotide
taacaaaactcaccaatctctact

SEQ ID NO: 45 - DNA, 20 bp, Artificial Sequence
  Synthetic Oligonucleotide
ggttgggagggtaggtagga

SEQ ID NO: 46 - DNA, 24 bp, Artificial Sequence
  Synthetic Oligonucleotide
ttacctaaaatcaacaaaaaaccc

SEQ ID NO: 47 - DNA, 22 bp, Artificial Sequence
  Synthetic Oligonucleotide
gttgggtagttaggagtttggg

SEQ ID NO: 48 - DNA, 25 bp, Artificial Sequence
  Synthetic Oligonucleotide
ctctctcacctcctctaaatacctc

SEQ ID NO: 49 - DNA, 19 bp, Artificial Sequence
  Synthetic Oligonucleotide
ttagggtgtaggatggagg

SEQ ID NO: 50 - DNA, 19 bp, Artificial Sequence
  Synthetic Oligonucleotide
acractccccaaattctac

SEQ ID NO: 51 - DNA, 245 bp, Homo Sapiens
ggccgcccagccccggggtttgcgaaggtccggggtaggagggctgcagcctgcgcgacgcggcgggaggctacccgggggcgatgggaaggcgggcgcagtcgacccaagggtggagaagagggaaggcgaaggacgcgcgttcccgggctcgtgaccgccagcggcccggggaacccgctcccagacagactcggagagatggcaggcggaagacaccggcgcgtcgtgggcaccctccacct

SEQ ID NO: 52 - DNA, 219 bp, Homo Sapiens
agaagcagtcagcccagggctctcggatgcagggagcctgggcccaaacagcagcttccggagtcggaaggagctgaggaagaagactgactgaaggagcttgcgacttttccgcctcggcaaccggacccagcagcaagcaggacgggcggcgctctgctactggtcccgttaagccagagtagcccaagccctgaagtcactgctcatccggaatgg

SEQ ID NO: 53 - DNA, 248 bp, Homo Sapiens
ccttccttttcctaatccctggctgcttattttagccgcttctccgccccgccgggaataccattcggatcttagtccaggtagatctgacgtcaagagatggctttcgtcgatttgacgtgtaaacactcatttccattctggctgggaagggctggggctccactcagcctggagaccgaagcgcttcactgagcgctcgccgccgcccagcctctcctctcgcgcctcctagctcttcgcagagc

SEQ ID NO: 54 - DNA, 1352 bp, Homo Sapiens
ggccaaggggggactgggaatcctggagggccaggtctgggggagagttaggaggtcgtgaatttggacttggatgcttgttgaagctgatgcatcttgaggacatttgtgggacacataggctgggtcagggctgaaagaggtgctggttatggccgggggcagggactcatgcctgtaatcccaacagcccaggaggatgagacaggaggaatgcttgaggccaaaatttcgaggccggaagttccagacgagcctgggcaacacagcaagaccctgtctctagaaaaggaaagaaagaaccgctggttgtggaagccagccatggcccagagctcagcagtgtaggagaggagggtgcgggcctgagagaggcagcaggcttggctggagaggcagaaggaaaaccaaggcaggagagtgtcctggaagctgggagaaggcagagggagatcgaggcttactttctggctgaggggcctagggtgagtcactttgggaggcttgatttcctcctctgtgaaatgggcaacacacctacccttgcccacctcacccccctcaccccgtcgacgtcgagtgaggagcagctgtgaggaggaggaagctttgcacagacgtgaggacggccccgggcgcctctaccccggggacacccccccccccaggagatggaaccccgagggcacggcgtggcggcccatgcgggcagctcgcgttaggacccagcggctccggggctggagggcgggcgccagccccgtcgggggcccggagggggactcggagcgggccaaggggcggctccggcgggcggactcggagcgggcggcggagtgacccggacaggtaggcgcgggcctggcgcttgggggccccggggggcggtcaggggcgtcggcgggtcccccggcgcgggggcgagggtgccaggctctctgggtcctcgatcctggggcagccgcggaggttcctgaaccccgcgccttcagagcggaggccaagggggcacctggaaagtttggggttgctctgcagagagaacgcgaattgggaagggcggggagcccgagtttggggggagccagaggtgaggtcgtcgggggatgggacatcttttgttctcccggggaggttggaatgtggcgggtggggtggggggggggggtccggtgggaatggccgagacagagacgcagagagacaaagagagatcagagacgggagcgcgagggcgcgcggagcaagtttccgcggctgattgttcccagctgtgcggcgctgcaacatctgggtttctcccgccgggacccctccccctgagcccggctctggagccggcagagccctcc

SEQ ID NO: 55 - DNA, 319 bp, Homo Sapiens
gtgctggacgcggacacatgatccgagggaccctgctgggtggaactaagaaagtccagcagactgtgcacgctcctgtccccactcacaggcccacgcagcgaggggggcccctcttctgtgtgcgtctggaaggtcgctgcccagggaggaaatgcctttaaccggcgtggggccgggcagctgcaggaggcagatcatccgggctctgtgcctcttgctacttctcctccacgccggctctgccaagaatatctggaaacgggcattgcctgcgaggctggccgagaaatcccgtgtaagtgcctgggatggggag

SEQ ID NO: 56 - DNA, 342 bp, Homo Sapiens
cggctcacttttccacatcacttcacagttacatttggcaggcaggcgggctcgcacgccggagcgctcagcccagaattagtggatttatttggaatctccctgcctcctccaagctccgccgccgccgccggcttgtgcatagacggtagcgtcaagacaccagctgcactttgggttgcggacacccagagcccagcgcgcccgccctgcgtctccgcaccaagccctgtagctccgagaggcatgaacggaatccggaggcgcctgcctagcgagcgaggaccggtcggcgcttgccgccctcgggagctaaccccgagcacctccagccatttgtga

SEQ ID NO: 57 - DNA, 518 bp, Homo Sapiens
atccgcctacaaatatagcctgttagcgcacaggcacgggaggcttcttccggctcggccgtgctgctcagcgggtgtaaacagcctctgcggtgtaaacagcctctgcggtgtaaacagccgcggcgggcaggccaggactgtcagtgcccgcccctcggggcaggtaagcgctgggcgtccgaggtggccggggggcgggagcgggggccttgcgctccccgtgggaggacgcgggcggatggccggtgccccgggcgctcagttgtttaatccttcctctgtgccttggtgacctgcaatctgccggagcgacaggggtaaacgtccctaaccggacagcggctggggcttaggcatccaccgttatctgaggtccaggactatggtgggaaggcgagaaaggagagagcgtggggaggcaggggtccccgcgagtgaatcttgctccggcttagcagttcccctgcgcgtactgctccggcgcccaaggctgagcccaaagctttctggtccaa

SEQ ID NO: 58 - DNA, 70 bp, Homo Sapiens
gggaacccgctcccagacagactcggagagatggcaggcggaagacaccggcgcgtcgtgggcaccctcc

SEQ ID NO: 59 - DNA, 69 bp, Homo Sapiens
gggaatttgtttttagatagatttggagagatggtaggtggaagatattggtgtgttgtgggtattttt

SEQ ID NO: 60 - DNA, 69 bp, Homo Sapiens
  misc_feature (25)..(25): n is a, c, g, or t
gggaattcgtttttagatagattcngagagatggtaggcggaagatatcngcgcgtcgtgggtattttt

(19) United States
(12) Patent Application Publication
(10) Pub. No.: US 2010/0143899 A1
(43) Pub. Date: Jun. 10, 2010

(54) METHODS AND PRODUCTS FOR DIAGNOSING CANCER

(75) Inventors: Marcus Bosenberg, Guilford, CT (US); Viswanathan Muthusamy, Woodbridge, CT (US); Sekhar Duraisamy, Brookline, MA (US)

Correspondence Address: WOLF GREENFIELD & SACKS, P.C., 600 ATLANTIC AVENUE, BOSTON, MA, 02210-2206, US

(73) Assignee: University of Vermont and State Agricultural College, Burlington, VT (US)

(21) Appl. No.: 12/312,807

(22) PCT Filed: Nov. 29, 2007
(86) PCT No.: PCT/US07/24553

§ 371 Date: Dec. 21, 2009

Related U.S. Application Data
(60) Provisional application No. 60/681,755, filed on May 17, 2005.

(51) Int. Cl. C12Q 1/68

(52) U.S. Cl. 435/6

(57) ABSTRACT

The present invention relates to methods and kits for the detection and diagnosis of cancer or precancerous conditions. Methylation of specific genes has been identified as indicative of cancer and methods of the invention, in part, relate to the detection of methylation levels in cells as a determination of cancer or a precancerous condition in the cell.

### RELATED APPLICATIONS

This application claims the benefit under 35 U.S.C. §119(e) of U.S. provisional application Ser. No. 60/861,755, filed Nov. 29, 2006, the content of which is incorporated by reference herein in its entirety.

### FIELD OF THE INVENTION

The invention pertains to the field of diagnosis of cancer and precancerous conditions.

### BACKGROUND OF THE INVENTION

Methylation of bases in DNA serves a number of cellular functions. In bacteria, methylation of cytosine and adenine residues plays a role in the regulation of DNA replication and DNA repair. DNA methylation also constitutes part of an immune mechanism that allows these organisms to distinguish between self and non-self DNA. In mammalian species, DNA methylation occurs only at cytosine residues, and specifically at cytosine residues that lie next to a guanosine residue, i.e., within the sequence CG. Methylation of DNA is carried out by DNA methyltransferases (sometimes called methylases). Generally both DNA strands can accept methyl groups at opposing CG sites. Replication of these strands yields a hemi-methylated state which is recognized by a class of maintenance methyltransferases capable of restoring full methylation to both strands. Most CG sites in the genome are methylated except for those in CpG ‘islands’ which remain methylation-free. CpG ‘islands’ are rich in CG sites and are often found near coding regions within the genome (i.e., genes). If a CpG island region of a gene is methylated the expression of that gene may be repressed.

Alterations in DNA methylation are one manifestation of the genome instability characteristic of human tumors. A hallmark of human carcinogenesis is the loss of normal constraints on cell growth resulting from genetic alterations in the genes that control cell growth. The consequences of such mutations include the activation of positive growth signals and the inactivation of growth inhibitory signals. Gene function can be lost through mutation or deletion. An alternative mechanism by which gene function can be lost is aberrant DNA methylation. Accordingly, such methylation events can be viewed as key steps in both the initiation and progression of cancer.

### SUMMARY OF THE INVENTION

The present invention relates to methods for the detection, diagnosis, and monitoring of cancer or a precancerous condition in a cell or subject. In some embodiments, the invention relates to methods for the detection, diagnosis, and monitoring of melanoma in a cell or subject. Methods and kits of the invention are highly sensitive and thus permit the identification of abnormal methylation of one or more genes of the invention in very rare cell events as well as in more common cell events and conditions. Methods and kits of the invention permit quantitative and qualitative evaluation of the methylation state of genes associated with cancer or precancerous conditions, thus permitting assessment of cancer and/or precancerous conditions in cells and subjects.

Methods of the invention, in part, include determination the methylation state in a cell or sample of one or more of the genes QPCT, LXN, PCSK1, MFAP2, WFDC1, CDH8 and LRRC2, which have now been identified as hypermethylated in cancer and/or precancerous conditions. The methylation state of one or more of these genes may be used to diagnose and monitor cancer or a precancerous condition in a cell or sample. In some aspects, the invention may include assaying the methylation state of one or more of the QPCT, LXN, PCSK1, MFAP2, WFDC1, CDH8 and LRRC2 genes, and assaying of the methylation state of one or more additional genes, some of which have been previously identified as being abnormally methylated in cancer or precancerous conditions.

According to one aspect of the invention, methods for diagnosing cancer or a precancerous condition in a subject are provided. The methods include determining a level of methylation of one or more of the genes QPCT, LXN, PCSK1, MFAP2, WFDC1, CDH8 and LRRC2 in a sample from a subject, comparing the level of methylation of the one or more genes in the sample to a control level of methylation of the one or more genes, wherein a higher level of methylation of the one or more genes in the sample compared to the control level of methylation is diagnostic for cancer or precancerous condition in the subject. In some embodiments, the level of two or more of the genes is determined in the sample. In certain embodiments, the methods also include determining the level of methylation of one or more of the genes CYP1B1, COL1A2, GDF15, RARB, TM, 3-OST-2, RASSF1A, ACS/TMS1, BST2, DNAJC15, CDKN1c, MGMT, SYK, MiB1, HOXB13, PTGS2, DAPK, APC, p16^{INK4A}, p27^{Kip1}, PRDX2, PYCARD, CDKN2A, CDKN1B and DAL1. In some embodiments, the methylation of the one or more genes is located on CpG islands of the one or more genes. In some embodiments, the methylation of the one or more genes is located on a nucleotide sequence of SEQ ID No. 51-57. In some embodiments, the cancer is melanoma. In certain embodiments, the cancer is metastatic cancer. In some embodiments, the control level of methylation of the one or more genes is the level of methylation of the one or more genes in a non-cancerous cell. In some embodiments, the non-cancerous cell is a cultured melanocyte. In certain embodiments, the sample is a fluid sample. In some embodiments, the fluid sample is a blood sample. In certain embodiments, the sample is a tissue sample. In some embodiments, the tissue sample is a lymph node sample. In some embodiments, the level of methylation of the one or more genes in the sample is at least 1%, 10%, 20%, 50%, 100%, 200%, 400% or 1000% higher than the control level of methylation of the one or more genes. In some embodiments, the level of methylation of the one or more genes in the sample is at least 400% higher than the control level of methylation of the one or more genes. In certain embodiments, the level of methylation of the one or more genes is determined by methylation-specific PCR, methylation-inhibitor analysis, methylation sensitive restriction analysis, sequencing of bisulfite modified DNA, methylation-sensitive single nucleotide primer extension, MethyLight analysis, pyrosequencing, or combined bisulfite restriction analysis. In some embodiments, methylation-inhibitor analysis includes adding a DNA methylation inhibitor to the sample and monitoring the change in expression level of the one or more genes upon addition of the DNA methylation inhibitor, wherein if the expression level of the one or more genes increases, the one or more genes have an increased level of methylation. In some embodiments, the methylation inhibitor is 5-aza-2′-deoxycytidine. In certain embodiments, the methods also include isolating a nucleic acid from the sample and analyzing the nucleic acid on a nucleic acid microarray. In some embodiments, the methods also include analyzing the sample for one or more mutated genes. In some embodiments, the one or more mutated genes is p16^{INK4A} or p14^{ARF}. In some embodiments, the methods also include analyzing the sample for one or more chromosomal instability loci. In certain embodiments, the one or more chromosomal instability loci include 6q, 8p, 9p, 10, 13, 21q, 6p, 7, 8q, 11q, q3, 17q and 20q. In some embodiments, the subject is asymptomatic for cancer.

According to another aspect of the invention, methods for determining onset, progression, or regression, of cancer in a subject are provided. The methods include determining a level of methylation of one or more of the genes QPCT, LXN, PCSK1, MFAP2, WFDC1, CDH8 and LRRC2 in a sample obtained from the subject, determining the level of methylation of the one or more genes in a second sample obtained from the subject at a later time than the first sample was obtained, comparing the level of methylation of the one or more genes in the first sample with the level of methylation of the one or more genes in the second sample, wherein a lower level of methylation of the one or more genes in the first sample compared with the level of methylation of the one or more genes in the second sample indicates onset or progression of cancer in the subject, and wherein a higher level of methylation of the one or more genes in the first sample compared with the level of methylation of the one or more genes in the second sample indicates regression of cancer in the subject. In some embodiments, the level of two or more of the genes is determined in the sample. In certain embodiments, the methods also include determining the level of methylation of one or more of the genes CYP1B1, COL1A2, GDF15, RARB, TM, 3-OST-2, RASSF1A, ACS/TMS1, BST2, DNAJC15, CDKN1c, MGMT, SYK, MiB1, HOXB13, PTGS2, DAPK, APC, p16^{INK4A}, p27^{Kip1}, PRDX2, PYCARD, CDKN2A, CDKN1B and DAL1. In some embodiments, the methylation of the one or more genes is located on CpG islands of the one or more genes. In some embodiments, the methylation of the one or more genes is located on a nucleotide sequence of SEQ ID No. 51-57. In some embodiments, the cancer is melanoma. In certain embodiments, the cancer is metastatic cancer. In some embodiments, the control level of methylation of the one or more genes is the level of methylation of the one or more genes in non-cancerous cells. In some embodiments, the non-cancerous cells are cultured melanocytes. In certain embodiments, the sample is a fluid sample. In some embodiments, the sample is a blood sample. In certain embodiments, the sample is a tissue sample. In some embodiments, the sample is a lymph node sample. In some embodiments, the level of methylation of the one or more genes is at least 1%, 10%, 20%, 50%, 100%, 200%, 400%, or 1000% higher in the sample than in the control. In certain embodiments, the level of methylation of the one or more genes is at least about 400% higher in the sample than in the control. In some embodiments, the level of methylation of the one or more genes is determined by methylation-specific PCR, methylation-inhibitor analysis, methylation sensitive restriction analysis, sequencing of bisulfite modified DNA, methylation-sensitive single nucleotide primer extension, MethyLight analysis, or combined bisulfite restriction analysis. In some embodiments, methylation-inhibitor analysis includes adding a DNA methylation inhibitor to the sample and monitoring the change in expression level of the one or more genes upon addition of the DNA methylation inhibitor, wherein if the expression level of the one or more genes increases, the one or more genes have an increased level of methylation. In certain embodiments, the methylation inhibitor is 5-aza-2′-deoxycytidine. In some embodiments, the method also includes isolating a nucleic acid from the sample and analyzing the nucleic acid on a nucleic acid microarray. In some embodiments, the method also includes analyzing the sample for one or more mutated genes. In certain embodiments, the one or more mutated genes is p16^{INK4A} or p14^{ARF}. In some embodiments, the method also includes analyzing the sample for one or more chromosomal instability loci. In some embodiments, the one or more chromosomal instability loci include 6q, 8p, 9p, 10, 13, 21q, 6p, 7, 8q, 11q, q3, 17q and 20q. In certain embodiments, the subject is undergoing treatment for cancer. In some embodiments, the subject has been diagnosed with cancer. In some embodiments, the subject is asymptomatic for cancer.

According to yet another aspect of the invention, kits for diagnosing cancer or a precancerous condition in a subject are provided. The kits include one or more containers, each container containing a nucleic acid for performing methylation-specific PCR to determine the level of methylation of one or more of the genes QPCT, LXN, PCSK1, MFAP2, WFDC1, CDH8, and LRRC2, and the kit also includes instructions for using the nucleic acids to determine the level of methylation of the one or more genes. In certain embodiments, the cancer is melanoma.

According to yet another aspect of the invention, kits for diagnosing cancer or a precancerous condition in a subject are provided. The kits include a container containing a DNA methylation-inhibitor and instructions for using the DNA methylation inhibitor to perform methylation-inhibitor analysis to determine the level of methylation of the one or more of the genes QPCT, LXN, PCSK1, MFAP2, WFDC1, CDH8 and LRRC, and the kit optionally includes a control nucleic acid. In some embodiments, the kit also includes an additional one or more containers, each containing a reagent to perform methylation inhibition analysis to determine the level of methylation of the one or more of the genes QPCT, LXN, PCSK1, MFAP2, WFDC1, CDH8 and LRRC2. In some embodiments, the kit also includes a nucleic acid microarray, and instructions for using the nucleic acid microarray to determine the level of methylation of the one or more of the genes QPCT, LXN, PCSK1, MFAP2, WFDC1, CDH8 and LRRC2. In certain embodiments, the cancer is melanoma.

According to another aspect of the invention, methods for screening for a candidate therapeutic agent for treatment of cancer are provided. The methods include contacting an agent with a sample that includes cancer cells, determining the level of methylation of one or more of the genes QPCT, LXN, PCSK1, MFAP2, WFDC1, CDH8 and LRRC2, and comparing the level of methylation of the one or more genes in the sample to the level of methylation of the one or more genes in a control sample, wherein a lower level of methylation of the one or more genes in the sample contacted with the agent compared to the control level, indicates that the agent is a candidate therapeutic agent for treatment of cancer.

In some embodiments, the cancer is melanoma. In certain embodiments, the level of methylation of the one or more genes is determined by methylation-specific PCR, methylation-inhibitor analysis, methylation sensitive restriction analysis, sequencing of bisulfite modified DNA, methylation-sensitive single nucleotide primer extension, MethyLight analysis, pyrosequencing, or combined bisulfite restriction analysis. In some embodiments, the sample includes cultured cells. In certain embodiments, the sample is a sample obtained from a subject.

According to yet another aspect of the invention, methods for monitoring response to a cancer treatment in a subject with cancer are provided. The methods include detecting a to level of methylation of one or more of the genes QPCT, LXN, PCSK1, MFAP2, WFDC1, CDH8, and LRRC2 in a first sample obtained from the subject, administering the cancer treatment to the subject, detecting the level of methylation of the one or more genes in a second sample, wherein the second sample is obtained from the subject after treatment and at a time later than the first sample, and comparing the level of methylation of the one or more genes in the first sample with the level of methylation of the one or more genes in the second sample, wherein a lower level of methylation of the one or more genes in the second sample than in the first sample indicates that the subject is responsive to the cancer treatment. In some embodiments, the cancer is melanoma. In certain embodiments, the treatment includes chemotherapy, radiation, and/or surgical therapy. In some embodiments, the level of methylation of the one or more genes is determined by methylation-specific PCR, methylation-inhibitor analysis, methylation sensitive restriction analysis, sequencing of bisulfite modified DNA, methylation-sensitive single nucleotide primer extension, MethyLight analysis, pyrosequencing, or combined bisulfite restriction analysis.

According to yet another aspect of the invention, methods for selecting a course of treatment of a subject having or suspected of having cancer are provided. The methods include detecting a level of methylation of one or more of the genes QPCT, LXN, PCSK1, MFAP2, WFDC1, CDH8I and LRRC2 in a sample obtained from a subject, comparing the level of methylation of the one or more genes to a control level of methylation of the one or more genes, determining the stage and/or type of cancer of the subject based at least in part on the difference in the level of methylation of the one or more genes in the sample compared to the control level of methylation, and selecting a course of treatment for the subject appropriate to the stage and/or type of cancer of the subject. In some embodiments, the cancer is melanoma. In certain embodiments, the level of methylation of the one or more genes is determined by methylation-specific PCR, methylation-inhibitor analysis, methylation sensitive restriction analysis, sequencing of bisulfite modified DNA, methylation-sensitive single nucleotide primer extension, MethyLight analysis, pyrosequencing, or combined bisulfite restriction analysis. In some embodiments, the treatment includes chemotherapy, radiation, and/or surgical therapy.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments, and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF THE DRAWINGS

FIG. 1 shows a schematic diagram of expression changes induced by 5AzadC treatment in melanoma cell lines. FIG. 1A shows changes in expression of selected candidate genes upon 48 h 5AzadC treatment validated by quantitative RT-PCR analysis. FIG. 1B shows expression of the genes in untreated melanoma cell lines relative to primary melanocytes by quantitative RT-PCR. FIG. 1C provides microarray profile data of genes showing significant expression changes upon 48 h 5AzadC treatment. Genes were ordered by median expression change relative to corresponding untreated cells.

FIG. 2 shows panels and a graph indicating methylation status of candidate melanoma associated genes. FIG. 2A shows the methylation status of candidate genes in nine melanoma cell lines. Black shading box indicates presence of methylation in the promoter region CpG island of the gene. *Indicates additional cell lines used in the bisulfite sequencing analysis originally not included in the microarray screening. FIG. 2B shows the methylation status of the candidate tumor suppressor genes in a panel of twenty melanoma tumor tissues. Black shading box indicates presence of methylation in the promoter region CpG island of the gene. FIG. 2C is a comparison of frequency of methylation of the candidate genes in tumors versus cell lines.

FIG. 3 provides panels indicating methylation status of individual CpG sites of target genes determined by bisulfite sequencing. The methylation status of each gene is indicated in the following panels: FIG. 3A, LXN; FIG. 3B, BST2; FIG. 3C, GDF15; FIG. 3D, WFDC1; FIG. 3E, CDKN1C; FIG. 3F, PTGS2; FIG. 3G, PCSK1; FIG. 3H, LRRC2; FIG. 3I, CDH8; FIG. 3J, QPCT; FIG. 3K, HOXB13; FIG. 3L, DNAJC15; FIG. 3M, COL1A2; FIG. 3N, CYP1B1; FIG. 3O, DAL1; FIG. 3P, MFAP; and FIG. 3Q, SYK, respectively. Dark shading indicates presence of methylation. The location of each CpG site relative to the transcriptional start site of the gene is indicated at the top of each panel.

FIG. 4 provides histograms showing results of analysis of expression changes following 5AzadC treatment by quantitative RT-PCR analysis. FIG. 4A, FIG. 4B, FIG. 4C, FIG. 4D, FIG. 4E, and FIG. 4F show results for HOXB13, LXN, GDF15/PLAB, SYK, DNAJC15/MCJ, and EPB41L3/DAL1, respectively. Shaded circles on top of the bars indicate presence of methylation in the promoter region of the gene in the corresponding cell line.

FIG. 5 shows electropherograms depicting bisulfite sequencing of the QPCT promoter region. FIG. 5A represents the original sequence for comparison (SEQ ID NO:58). FIG. 5B represents the sequence of the QPCT promoter region in primary cultured melanocytes (SEQ ID NO:59); FIG. 5C represents the sequence of the QPCT promoter region in the untreated MelJuSo melanoma cell line (SEQ ID NO:60); and FIG. 5D represents the sequence of the QPCT promoter region in the MelJuSo melanoma cell line after 48 h treatment with 5AzadC (SEQ ID NO:59), demonstrating demethylation of the promoter region.

FIG. 6 provides histograms and a digitized image of a Western blot showing SYK expression in melanoma cell lines and melanoma tumor samples. FIG. 6A shows results of quantitative RT-PCR analysis of SYK mRNA expression in melanoma cell lines compared to primary human melanocytes. FIG. 6B shows a Western blot of SYK expression in melanoma cell lines. FIG. 6C shows results of quantitative RT-PCR analysis of SYK mRNA expression in tumor samples compared to melanocytes.

FIG. 7 provides histograms and digitized images of Western blots demonstrating SYK and HOXB13 expression in Xenografted tumors. FIG. 7A shows average weights of tumors formed by vector and SYK transfected clone in nude mice. SYK 7 represents grouping of smaller tumors (<80 mg) SYK7* represents grouping of larger tumors (>80 mg) at the endpoint of experiment. FIG. 7B shows results of RT-PCR analysis of SYK expression in tumors formed by the SYK7 clone. Expression of SYK is present in the clonal transfected cell line (SYK7, FIG. 7D) and in smaller tumors (SYK7) but is lost in large tumors (STK7* above). FIG. 7C shows results of RT-PCR analysis of HOXB13 expression in tumors formed by HOXB13 clones. FIG. 7D shows expression of SYK in stably transfected clonal cell lines compared to primary human melanocytes and the parental MelJuSo cell line. FIG. 7E shows expression of HOXB13 in stably transfected clonal cell lines compared to primary human melanocytes and the parental MelJuSo cell line.

FIG. 8 shows graphs, digitized images of expression assays, and digitized images of tumor growth. FIG. 8A-D show tumor suppressor characteristics of HOXB13 in-vitro and in-vivo; FIG. 8A shows results of an in-vitro proliferation assay of HOXB13 transfected MelJuSo clones compared to vector controls (pTRE). The results presented are an average of 3 replicates counted on a flow cytometer (p-value 120 h<0.01). FIG. 8B shows representative plates of a colony formation assay comparing vector and HOXB13 transfected MelJuSo clones. FIG. 8C shows results of tumor growth of subcutaneous xenografts of vector and HOXB13 transfected clones in nude mice. The results presented here represent average of four subcutaneous injections (p-value 10 weeks <0.05). FIG. 8D shows images of representative tumors formed by vector controls and HOXB13 transfected MelJuSo cell line. FIGS. 8E-H show tumor suppressor characteristics of SYK in-vitro and in-vivo. FIG. 8E shows results of an in-vitro proliferation assay of SYK transfected MelJuSo clones compared to vector controls. The results represent an average of three replicates counted on a flow cytometer (p-value 120 h<0.01). FIG. 8F shows representative plates of a colony formation assay comparing vector and SYK transfected MelJuSo clones. FIG. 8G shows tumor growth of subcutaneous xenografts of vector and SYK transfected clones in nude mice showing tumor kinetics over a 10 week period. The results presented here represent average of four subcutaneous injections (p-value 10 weeks <0.05). FIG. 8H shows average weights of tumors formed by vector control and HOXB13, SYK transfected clones at 10 weeks following xenografting. The results represent an average of 8 tumors each of vector, HOXB13 transfected clones and 4 tumors of SYK transfected clone (p-value <0.01).

FIG. 9 provides histograms and digitized images of Western blots demonstrating HOXB13 expression in melanoma cell lines and melanoma tumor samples. FIG. 9A shows results of quantitative RT-PCR analysis of HOXB13 mRNA expression in melanoma cell lines compared to primary human melanocytes. FIG. 9B shows a Western blot of HOXB13 expression in melanoma cell lines. FIG. 9C shows results of quantitative RT-PCR analysis of HOXB13 mRNA expression in tumor samples compared to melanocytes.

FIG. 10 is a schematic diagram of a kit for diagnosing cancer (10=kit, 12=component for determining the level of DNA methylation determination; 14=additional components; 20=instructions).

### DETAILED DESCRIPTION

Cancers may arise from any number of cellular perturbations in a cell. Most of these perturbations take the form of a genetic mutation at the genomic DNA level. Genetic mutations can in turn manifest their effects in a number of ways including alterations in expression levels and/or function of an mRNA or a polypeptide. The end result is an uncontrolled growth of the mutated population of cells as a result of increased proliferative rates, decreased apoptotic rates, and/or failure to respond to normal growth-control signals.

Gene loci that are altered in the progression of such disorders are not always the primary or direct target of the initial mutation. Rather, a mutation may exist in a genomic locus that encodes an “upstream” factor. Mutation of the upstream factor may not produce a malignant phenotype to a cell by itself, but the mutation of the upstream factor may impact one or more “downstream” factors, the genomic locus of which remains essentially wild type.

One such upstream factor is a factor capable of methylating genomic sequences. Abnormal methylation of genomic loci has been reported to cause altered expression levels from that genomic locus. The mammalian genome is widely methylated except for regions rich in CG dinucleotides (e.g., CpG islands), which in normal cells are undermethylated as compared to the rest of the genome. DNA methylation is one form of epigenetic change and involves the covalent addition of a methyl group to cytosine residues in CpG dinucleotides by DNA methyltransferases. Abnormal methylation, particularly at CpG islands, may be accompanied by gene silencing and may be one mechanism responsible for the inactivation of several tumor suppressor genes in human cancers. The invention described herein is premised, in part, on the identification of genes that are abnormally methylated in cancer.

The discovery of specific genes that are hypermethylated facilitates analysis of cancer and cancer treatments. For example, it has been discovered that an increased level of methylation of certain genes may lead to gene activation (e.g., epigenic silencing). Epigenic silencing of a gene may prevent normal functioning of the gene, and if the normal function of the gene includes tumor suppression activity, the epigenic silencing resulting from hypermethylation (e.g., in CpG islands of the gene) may result in cancer. Modifying the amount of such hypermethylation of the genes of the invention may be useful to prevent and/or treat cancer. Compounds that decrease hypermethylation of one or more genes of the invention may result in more normal functioning of the genes and a corresponding prevention or treatment of cancer or of a precancerous condition. In addition, methods to assess hypermethylation in the genes of the invention may be used to monitor the onset, progression, and/or regression of cancer by monitoring levels of methylation in a cell or subject and determining the effect of a candidate therapeutic compound on the level of methylation of such genes. Such monitoring may also be used to assess the efficacy of treatments administered to an individual subject by monitoring the level of methylation of genes of the invention in a sample or subject before, during, and after administration of a treatment regimen (e.g., a therapeutic agent).

The present invention provides methylation assays for assessing a methylation state of a gene in a cell, tissue, and/or subject. As used herein the term “methylation assay” refers to any assay for determining the methylation state of a CpG dinucleotide within a sequence of DNA. As used herein, the term “methylation state” refers to the presence or absence of 5-methylcytosine (“5-mCyt”) at one or a plurality of CpG dinucleotides within a DNA sequence. The term “hypermethylation” refers to a methylation state that corresponds to an increased presence of 5-mCyt at one or a plurality of CpG dinucleotides within a DNA sequence of a test DNA sample, relative to the amount of 5-mCyt found at corresponding CpG dinucleotides within a normal control DNA sample. As used herein the term “hypomethylation” refers to the methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

Methods of the invention may include determining a level of methylation of CpG islands of genes of the invention in a sample and comparing the level to a control level as a measure of whether the amount of methylation is abnormal compared to the control level of methylation (e.g., the level of methylation in a control sample). Such comparisons may be useful for diagnosing cancer in a cell and/or subject and for assays to identify treatments for cancer and for the selection of treatment paradigms for subjects diagnosed with cancer. As used herein the term “CpG Island” refers to a contiguous region of genomic DNA that satisfies the criteria of (1) having a frequency of CpG dinucleotides corresponding to an “Observed/Expected Ratio”>0.6), and (2) having a “GC Content”>0.5. As used here in the term “GC Content” refers, within a particular DNA sequence, to the [(number of C bases+number of G bases)/band length for each fragment]. The term “observed/expected ratio” or O/E ratio” means the frequency of CpG dinucleotides within a particular DNA sequence, and corresponds to the [number of CpG sites/(number of C bases×number of G bases)]× band length for each fragment. CpG islands are typically, but not always, between about 0.2 to about 1 kb in length. CpG islands are readily identifiable by those of ordinary skill in the art using routine procedures. A CpG island sequence associated with a particular SEQ ID NO sequence of the present invention is that contiguous sequence of genomic DNA that encompasses at least one nucleotide of the particular SEQ ID NO sequence, and satisfies the criteria of having both a frequency of CpG dinucleotides corresponding to an Observed/Expected Ratio>0.6), and a GC Content>0.5.

The invention, in part, also includes nucleic acid sequences that include methylated CpG islands, compositions comprising nucleic acids that comprise methylated CpG islands that can be used to assess methylation status in a cell or subject.

It has been determined that the presence of hypermethylation of CpG islands of genes of the invention in cells is correlated with the presence of cancer in the cells. Genes that have now been identified as hypermethylated in cancer include: QPCT, LXN, PCSK1, MFAP2, WFDC1, CDH8, and LRRC2. These genes, as well as other genes disclosed herein, some of which have been previously identified as having increased levels of methylation in cancerous cells are referred to herein as cancer-associated genes and/or as “genes of the invention”. Genes of the invention include the QPCT, LXN, PCSK1, MFAP2, WFDC1, CDH8, LRRC2, CYP1B1, COL1A2, GDF15, RARB, TM, 3-OST-2, RASSF1A, ACS/TMS1, BST2, DNAJC15, CDKN1c, MGMT, SYK, MiB1, HOXB13, PTGS2, DAPK, APC, p16^{INK4A}, p27^{Kip1}, PRDX2, PYCARD, CDKN2A, CDKN1B and DAL1 genes. Sequences of exemplary CpG island regions of some genes of the invention including QPCT, LXN, PCSK1, MFAP2, WFDC1, CDH8, and LRRC2 are provided in Table 3. The invention relates, in part, to the use of one or more methylation assays for determining the methylation state of CpG islands of at least one or more of the QPCT, LXN, PCSK1, MFAP2, WFDC1, CDH8 and LRRC2 genes in cells, tissues, and/or subjects to assess the cancer status of the cell, tissue, and/or subject.

Methods and kits of the invention are highly sensitive and may permit the identification of abnormal methylation of a gene of the invention in very rare cell events. Thus, the methods may be as sensitive as to permit detection of having abnormal methylation of one or more genes of the invention that are as rare as 1 abnormal cell per 10,000 cells, 1 abnormal cell per 100,000 cells, 1 abnormal cell per 1,000,000 cells etc. (including all values below and in between). Thus the methods of the invention permit detection and diagnosis of cancer and/or precancerous conditions based on very rare cellular events (e.g., isolated cancer cells circulating in peripheral blood and/or rare metastatic cells in a lymph node, etc.) These rare events may be clinically significant in the diagnosis and monitoring of cancer and/or precancerous conditions and may be detectable using methods and kits of the invention.

As used herein the term “cancer status” means the presence or absence of cancer, the stage of a cancer, and/or the detection of the presence, absence, or stage of a precancerous condition in a cell, tissue, and/or subject. An elevated level of methylation of a CpG island of one or more of the QPCT, LXN, PCSK1, MFAP2, WFDC1, CDH8 and LRRC2 genes versus a control level of methylation, may indicate that the tested cell or subject has cancer or a precancerous condition. As used herein, a precancerous condition is a condition that would not be clinically diagnosed as cancer but is indicative of an abnormality in the gene function in the cell, tissue, and/or subject that may be a precursor, or may lead to cancer. Examples of precancerous conditions, although not intended to be limiting include dysplasia, benign neoplasia, hyperplasia, atypical hyperplasia, metaplasia, carcinoma in situ, etc. The presence of hypermethylation of CpG islands of one or more of the QPCT, LXN, PCSK1, MFAP2, WFDC1, CDH8 and LRRC2 genes may be asymptomatic and yet indicate the subject will develop cancer or more advanced cancer if left untreated. Treatments for precancerous conditions may include surgery, chemotherapy, radiotherapy, etc. and treatments may be selected and their efficacy monitored using methods of the invention. Compounds and strategies for treating pre-cancerous conditions may be identified using assays and screening methods of the invention. In some embodiments, treatment of a precancerous condition may prevent or delay its development into cancer.

The invention, in part, provides methods for analyzing samples for features associated with the development of cancer or precancerous conditions, characterized in that the nucleic acid of at least one of QPCT, LXN, PCSK1, MFAP2, WFDC1, CDH8, LRRC2, and optionally at least one of CYP1B1, COL1A2, GDF15, RARB, TM, 3-OST-2, RASSF1A, ACS/TMS1, BST2, DNAJC15, CDKN1c, MGMT, SYK, MiB1, HOXB13, PTGS2, DAPK, APC, p16^{INK4A}, p27^{Kip1}, PRDX2, PYCARD, CDKN2A, CDKN1B and DAL1 is/are contacted with a reagent or series of reagents capable of distinguishing between methylated and non methylated CpG dinucleotides these genes.

As will be clear to those of ordinary skill in the art, the methylation state of only a portion of a gene of the invention that is present in a cell or subject need be assessed in a determination of the methylation state and cancer status of a cell or subject. The entire gene sequence need not be assayed. The term “genomic sequence” is used herein to refer to a region of a gene of the invention that may be assessed in a determination of the cancer status of a cell, tissue, or subject. A genomic sequence may correspond to a region of a gene of the invention that is assayed for methylation state and the genomic sequence may provide information on the methylation state of the gene of the invention that is sufficient to determine the gene's status as abnormally methylated (e.g., hypermethylated or hypomethylated) and the cancer or precancerous condition status of the cell or subject tested.

Detection of a methylation state of one or more genes of the invention (e.g., at least the QPCT, LXN, PCSK1, MFAP2, WFDC1, CDH8 and LRRC2 genes) may be used in combination with assessment of methylation state in one or more other genes of the invention that may also be abnormally methylated (hyper or hypomethylyated) in cancer and/or precancerous conditions. Additional genes whose methylation levels may be assessed in combination with one or more genes of the invention (e.g., QPCT, LXN, PCSK1, MFAP2, WFDC1, CDH8 and LRRC2) include, but are not limited to: CYP1B1, COL1A2, GDF15, RARB, TM, 3-OST-2, RASSF1A, ACS/TMS1, BST2, DNAJC15, CDKN1c, MGMT, SYK, MiB1, HOXB13, PTGS2, DAPK, APC, p16^{INK4A}, p27^{Kip1}, PRDX2, PYCARD, CDKN2A, CDKN1B and DAL1. Those of ordinary skill in the art will recognize that other genes that have increased methylation levels in cancer may also be used in combination with one or more of the genes identified herein for use in diagnostic methods and screening methods of the invention.

It will be understood that methods of the invention also encompass use of allelic variants of genes and/or sequence provided herein. Thus, there may be allelic variation in sequences of the genes assayed for methylation states using methods of invention, including wild-type gene sequences and/or mutant gene sequences. As used herein, the term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides with altered amino acid sequences. It will be understood by those of ordinary skill in the art that, such allelic variations may occur in full-length wild-type and mutant genes and may also occur in CpG island regions of genes. Thus, QPCT, LXN, PCSK1, MFAP2, WFDC1, CDH8 and LRRC2 genes, and other genes disclosed herein as useful in methods of the invention, may be allelic variants of wild-type genes or may be mutant gene sequences. Thus, determination of a methylation state of one or more genes of the invention may include determination of methylation state of wild-type and/or mutant form of the gene.

In some aspects, the invention may include examination of a full CpG island region of a gene of the invention, or may include assessment of a portion of a CpG island region of a gene that is sufficient to provide information on methylation state of the gene. For example, detection of hypermethylation in a portion of a CpG island of a gene of the invention can be used to ascertain the methylation state of the gene as a whole. Thus a portion of a CpG island region that is 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, or 900 nucleotides shorter (including all integers in between each of the foregoing integers) than the full length CpG island region of a gene may be assessed for its methylation state and the results used in the diagnosis of cancer or a precancerous condition as described herein.

Nucleic acid primers and DNA sequences and genes disclosed herein for use in assessing methylation states of genes for diagnose and assessment of cancer and precancerous conditions are meant to be exemplary. Those of ordinary skill in the art will be able to use routine procedures to recognize, design, and/or use alternative primers and/or sequences to assess methylation states of genes of the invention identified herein, including at least the QPCT, LXN, PCSK1, MFAP2, WFDC1, CDH8 and LRRC2 genes and CYP1B1, COL1A2, GDF15, RARB, TM, 3-OST-2, RASSF1A, ACS/TMS1, BST2, DNAJC15, CDKN1c, MGMT, SYK, MiB1, HOXB13, PTGS2, DAPK, APC, p16^{INK4A}, p27^{Kip1}, PRDX2, PYCARD, CDKN2A, CDKN1B and DAL1 genes or other genes useful in the methods of the invention. It will be understood that methods utilizing such alternative sequences (e.g. alternative primers, alternative portions of CpG regions of genes of the invention, etc) for the diagnosis and assessment of cancer and precancerous conditions in cells, tissues, and/or subjects are within the scope of the invention.

Methods for assaying methylation states of one or more genes of the invention may be carried out in cells from culture, cells in solution, and/or on samples obtained from subjects. As used herein, a subject is a human or a non-human animal, including, but not limited to a non-human primate, cow, horse, pig, sheep, goat, dog, cat, or rodent. In all embodiments, human subjects are preferred. Methods of the invention may be used to detect abnormal levels of methylation or expression products of genes in subjects not yet diagnosed with cancer. In addition, methods of the invention may be applied to subjects who have been diagnosed with cancer. A sample may comprise one or more cells. A sample may originate from a subject or culture, may be a lysate of a sample from a subject, and/or may be partially processed prior to use in methods of the invention. In some embodiments, a sample from a subject or culture may be processed to obtain DNA for use in assays for methylation as described herein. Thus, an initial step in an assay of methylation states that may be used use in methods and/or kits of the invention may include isolation of a genomic DNA sample from a cell, tissue, and/or subject. Extraction of DNA may be by any suitable means, including to routine methods used by those of ordinary skill in the art such as methods that include the use of detergent lysates, sonification, and vortexing with glass beads, etc. Once nucleic acids have been extracted from the sample, genomic double-stranded DNA may be used in further analysis of the methylation state of a sample. Methods of the invention may also include assessment of expression products of one or more genes of the invention in a sample.

As used herein, the term “sample” means any animal material containing DNA or RNA, such as, for example, tissue or fluid isolated from an individual (including without limitation plasma, serum, cerebrospinal fluid, lymph, tears, saliva and tissue sections) or from in vitro cell culture constituents. A sample containing nucleic acids can be drawn from any source and can be natural or synthetic. A sample containing nucleic acids may contain of deoxyribonucleic acids (DNA), ribonucleic acids (RNA), or copolymers of deoxyribonucleic acids and ribonucleic acids or combinations thereof. A sample may have been subject to purification (e.g. extraction) or other treatment. The term “sample may also refer to a “biological sample.”

As used herein, the term “biological sample” may refer to a whole organism or a subset of its tissues, cells or component parts (e.g. body fluids, including but not limited to blood, mucus, lymphatic fluid, synovial fluid, cerebrospinal fluid, saliva, amniotic fluid, amniotic cord blood, urine, stool, vaginal fluid, and semen, etc.). A “biological sample” may also refer to a homogenate, lysate, or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, stool, milk, blood cells, tumors, or organs, etc. A “biological sample” may also refer to medium, such as a nutrient broth or gel in which an organism or cell has been propagated, which contains cellular components, such as proteins or nucleic acid molecules.

Sample sources may include tissues, including, but not limited to lymph tissues; body fluids (e.g., blood, lymph fluid, etc.), cultured cells; cell lines; histological slides; tissue embedded in paraffin; etc. The term “tissue” as used herein refers to both localized and disseminated cell populations including, but not limited to: brain, heart, serum, breast, colon, bladder, epidermis, skin, uterus, prostate, stomach, testis, ovary, pancreas, pituitary gland, adrenal gland, thyroid gland, salivary gland, mammary gland, kidney, liver, intestine, spleen, thymus, bone marrow, trachea, and lung. Biological fluids include, but are not limited to, blood, lymph fluid, cerebrospinal fluid, tears, saliva, urine, and feces, etc. In preferred embodiments, a sample comprises a blood or lymph node sample. Invasive and non-invasive techniques can be used to obtain such samples and are well documented in the art. A control cell sample may include a cell, a tissue, or may be a lysate of either. In some embodiments, a control sample may be a sample from a cell or subject that is free of cancer and/or free of a precancerous condition. In some embodiments, a control sample may be a sample that is from a cell or subject that has cancer or a precancerous condition. Preferably, a biological sample corresponds to the amount and type of DNA and/or expression products present in a parent cell from which the sample was derived. If the sample is a melanoma tumor tissue sample or a melanoma cell line, cultured melanocytes may be, but need not be, used as a control.

As used herein, the term “cancer” refers to an uncontrolled growth of cells that may interfere with the normal functioning of the bodily organs and systems. Cancers that migrate from their original location and seed vital organs can eventually lead to the death of the subject through the functional deterioration of the affected organs. A metastasis is a cancer cell or group of cancer cells, distinct from the primary tumor location resulting from the dissemination of cancer cells from the primary tumor to other parts of the body. At the time of diagnosis of the primary tumor mass, the subject may be monitored for the presence of in transit metastases, e.g., cancer cells in the process of dissemination. Methods of the invention may be used to assess the status of primary and/or metastatic cancer.

As used herein, the term cancer, includes, but is not limited to the following types of cancer, breast cancer, biliary tract cancer, bladder cancer, brain cancer including glioblastomas and medulloblastomas; cervical cancer; choriocarcinoma; colon cancer; endometrial cancer; esophageal cancer, gastric cancer; hematological neoplasms including acute lymphocytic and myelogenous leukemia; T-cell acute lymphoblastic leukemia/lymphoma; hairy cell leukemia; chromic myelogenous leukemia, multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Other cancers will be known to one of ordinary skill in the art. In some embodiments of the invention, the cancer is melanoma.

Methods of the invention may be useful to assess characteristics of cancer in a cell or subject (e.g., for assessment of melanoma in a cell or subject). For example, characteristics such as whether a cancer is metastatic or non-metastatic and/or the present status or stage of a cancer may be assessed using methods of the invention. In general, cancer staging is based on a combination of clinical information obtained by physical examination, radiologic examination, etc. and pathogic information, which may be based on pathological examination of a tumor. Methods of the invention can provide pathological staging information about a cancer in a cell or subject. For example, an amount of methylation of one or more genes in a sample (e.g., from a tumor), or a particular pattern of methylation [e.g., a specific combination of two or more genes or regions of genes that are hypermethylated (or hypomethylated) in a sample] may be useful to assess the pathologic status of a cancer.

Genes that form the basis of the invention can be assessed singly or in sets of two or more (e.g, in panels). The use of a single gene assessment and/or use of a gene panel for multiple gene assessment allows for rapid, specific analysis of cancer or a precancerous condition that are associated with methylation states of included genomic sequences. A gene panel may be used with high efficiency for the diagnosis and monitoring of and the analysis of cancer, (e.g., melanoma) and/or precancerous conditions as described herein. A gene panel can also be used in screens for treatments for cancer and/or precancerous conditions. Thus, in some embodiments, the invention includes assays of methylation states of one or more CpG dinucleotides of more than one gene of the invention and in other embodiments, the invention includes assay of the methylation state of one or more CpG dinucleotides of a single gene of the invention for diagnosis, staging, and analysis of cancer in cells, tissues, and/or subjects.

Methods for assaying methylation states of genomic sequences of interest are well-known in the art. Examples of methods of assaying methylation states of genes of interest are provided herein, but are not intended to be limiting. Non-limiting examples of methylation assay methods are disclosed in patent documents such as U.S. Pat. No. 5,871,917; US Patent Application No. 20040081976; U.S. Pat. No. 6,893,820, U.S. Pat. No. 5,871,917; U.S. Pat. No. 5,786,146, U.S. Pat. No. 4,839,060, U.S. Pat. No. 6,200,756, U.S. Pat. No. 7,112,404, and U.S. Pat. No. 6,331,393, each of which is incorporated by reference herein in its entirety. Those of ordinary skill in the art will recognize that any suitable method that permits the assessment of the level of methylation of a CpG dinucleotide in a gene of the invention can be used in methods and kits of the invention.

### Assay of Methylation State

A level of methylation of one or more genes can be determined using a number of routine techniques and methods described in the art. Useful techniques for assay of methylation state in a sample include, but are not limited to: methylation-specific PCR (MSP) and other PCR based methods, methylation-inhibitor analysis, methylation-sensitive restriction analysis, sequencing of bisulfite-modified DNA, methylation-sensitive single nucleotide primer extension (Ms-SnuPE), MethyLight analysis, and combined bisulfite restriction analysis (COBRA), pyrosequencing, etc. Those of ordinary skill in the art will recognize that numerous additional methods for quantitatively and/or qualitatively assessing methylation status are routinely practiced in the art and may be used in methods and kits of the invention.

In one embodiment of the invention, a method for determining the level of methylation of one or more genes is methylation specific PCR (i.e., MSP), which is disclosed in U.S. Pat. No. 5,786,146 (incorporated by reference herein in its entirety). This method is based on the differential reactivity of cytosine and 5-methylcytosine with sodium bisulfite. In the presence of sodium bisulfite, cytosines are deaminated to uracils and 5-methylcytosines remain as cytosines. Primers used to amplify such bisulfite treated nucleic acid molecules are able to hybridize specifically either to the ‘unmethylated’ or the ‘methylated sequences. A multitude of parallel PCR reactions can be performed and analyzed on a gel simultaneously. Reaction vessels contain either methylation specific primers or primers specific for the unmethylated gene sequence, and thus an amplified product is formed only if the appropriate primers are present. It will be understood by those of ordinary skill in the art that in some embodiments, methylation-specific PCR may provide a quantitative and qualitative assessment of methylation, e.g., may include the use of MethyLight, TaqMan, or Sybr-based systems. In some embodiments, two reactions are performed for each sample, one with methylation specific primers; and one with non-methylation-specific primers.

Another PCR-based method for determining the level of methylation of one or more genes that can be used in the methods and kits of the invention, was reported by McGrew and Rosenthal and involves the use of ligation-mediated PCR (Biotechniques 1993, 15: 722-729), which is hereby incorporated by reference. Ligation-mediated PCR involves the measurement of conversion of large genomic DNA fragments to shorter DNA fragments as a function of demethylation. The cleavage of large genomic DNA is accomplished using pairs of non-isoschizometic enzymes, one of which is methylation specific. The digestion products are then amplified with ligation-mediated, radiolabeled PCR, and used as a measure of cleavage with the methylation sensitive restriction enzyme. Specifically, the ratio of the two amplified fragments is related to the degree of methylation at the particular restriction site. Internal control of the amplification reaction confers the quantitative aspect of the approach.

Methylation-inhibitor analysis comprises differential analysis of the level of methylation of one or more genes in samples treated with a DNA methylation inhibitor compared to untreated samples, and analysis of these samples using any suitable method for analysis of differential expression, including, but not limited to use of a nucleic acid microarray. A sample may include one or more genes that have a high level of methylation. Genes with a high level of methylation will have a low expression level. Treatment of the sample with a DNA methylation inhibitor results in inactivation or repression of the enzyme that methylates DNA, resulting in a lower level of methylation of the one or more genes than would be present in the absence of the inhibitor. A decrease in methylation of the one or ore genes will subsequently result in an increase of expression of the one or more genes. This difference in expression between a DNA methylation-inhibitor-treated sample and a control sample can be analyzed on using any suitable differential expression method, for example, using a nucleic acid microarray.

A DNA methylation inhibitor is an agent that directly or indirectly causes a reduction in the level of methylation of a nucleic acid molecule. DNA methylation inhibitors are well known and routinely utilized in the art and include, but are not limited to, inhibitors of methylating enzymes such as methylases and methyltransferases. Non-limiting examples of DNA methylation inhibitors include 5-azacytidine, 5-aza-2′deoxycytidine (also known as Decitabine in Europe), 5,6-dihydro-5-azacytidine, 5,6-dihydro-5-aza-2′deoxycytidine, 5-fluorocytidine, 5-fluoro-2′deoxycytidine, and short oligonucleotides containing 5-aza-2′deoxycytosine, 5,6-dihydro-5-aza-2′deoxycytosine, and 5-fluoro-2′deoxycytosine, and procainamide, Zebularine, and (−)-egallocatechin-3-gallate.

Another method that may be used to assay methylation states in the methods and kits of the invention is methylation-sensitive-restriction analysis. Methylation-sensitive restriction analysis is derived from the existence in nature of restriction enzymes that are methylation sensitive (i.e., these enzymes do not recognize restriction sites that contain methylated residues). For example, the restriction enzyme NotI recognizes the sequence containing 2 ‘CG’ dinucleotides. If either of the CG sites is methylated, the enzyme will not digest DNA. This fact has been utilized in the analysis of DNA methylation in genomic DNA. In this approach, DNA is digested with a methylation-sensitive restriction enzyme and then electrophoresed on an agarose gel that separates DNA based on its size. The DNA is then transferred to a membrane and hybridized to a detectably labeled probe (e.g., a radiolabeled or fluorescently labeled probe), as is routinely done in a Southern analysis. Based on the sizes of bands that hybridize to the probe, the digested, and therefore unmethylated, DNA can be distinguished from the undigested, and therefore methylated, DNA. Other methylation-sensitive enzymes include, but are not limited to: SacII, EagI, SmaI, That, HpaII, all of which are commercially available.

Genomic sequencing of bisulfite modified DNA is another method for determining the level of methylation of one more genes. Like other methylation assay methods described herein, this method is based on the differential reactivity of cytosine and 5-methylcytosine with sodium bisulfite. In this approach however, primers are designed to avoid potential methylation sites (e.g., CG dinucleotides) and a non-specific PCR is performed in order to amplify all alleles equally. Following amplification, the PCR product is sequenced directly, or can be subcloned into a plasmid and individual subclones sequenced. The sequence of amplified product is compared to that of non-bisulfite treated DNA. CG dinucleotides present in the non-bisulfite-treated sample that read as TG as a result of bisulfite treatment were unmethylated in the original sample, and those that continue to read as CG even after bisulfite treatment were originally methylated. This approach is quantitative to the extent that it provides an absolute number of methylated residues in a particular nucleic acid sequence.

Cytosine methylation can also be measured using an approach that combines automated genomic DNA sequencing and GENESCAN analysis. This approach has been reported by Paul et al. (Biotechniques 1996, 21:126-133), which is hereby incorporated by reference. This technique also requires bisulfite treatment and PCR amplification of DNA. Cloning and sequencing of the modified and amplified products is then performed to determine the methylation of individual DNA molecules. The sequencing of the entire population of amplified products provides the average methylation status over the population, and thus may not be appropriate if the methylation status of individual molecules is desired. By employing fluorescence-based automated genomic sequencing, Paul et al. were able to directly quantitate methylation status of any cytosine residue in a DNA molecule. The technique involves sequencing only cytosine and thymine residues of modified and amplified DNA and using fluorescent dyes to identify and visualize signals from these residues. GENESCAN analysis is then performed to estimate methylation at every cytosine in a rapid and accurate manner. The approach permits a rapid overview of DNA methylation profiles for a number of DNA molecules.

Another method for assaying methylation state in a sample is Methylation-sensitive single nucleotide primer extension (Ms-SnuPE). Ms-SnuPE has been reported by Gonzalgo et al. as a method for rapid quantitation of altered methylation patterns at specific sites, particularly CpG sites, in the genome (Nucl. Acids Res. 1997, 25: 2529-2531), which is hereby incorporated by reference. The approach relies on methylation-sensitive single nucleotide primer extension and involves bisulfite treatment of DNA followed by single nucleotide primer extension. It does not involve restriction enzyme analysis. Briefly, genomic DNA is first reacted with sodium bisulfite to convert methylated cytosine to uracil without modification of 5-methylcytosine, as in other approaches described herein. The bisulfite-treated DNA is then amplified using PCR primers specific for bisulfite-converted DNA. The amplified product is then used as a template for methylation analysis at the CpG site(s) of interest. The method is amenable to the analysis of small amounts of DNA. Like MSP, bisulfite sequencing, COBRA and most all other PCR based strategies, Ms-SNuPE can be used in the analysis of microdissected pathology sections and other samples.

MethyLight analysis is another PCR-based technique that can be used in methods and kits of the invention. MethyLight analysis includes analysis of bisulfite-treated DNA. Bisulfite treatment of DNA results in conversion of unmethylated cytosines to uracil but leaves methylated cytosines unaffected. Detectable-label-based PCR (e.g., fluorescence-label based PCR) may be subsequently performed with primers that either overlap CpG methylation sites or primers that do not overlap any CpG sequences. Quantitative sequence discrimination can occur either at the level of the PCR amplification process or at the probe hybridization process or both. The technique is described in detail in Eads et al. (Nucleic Acid Res. 2000, 28: e32), which is hereby incorporated by reference.

Another method of assaying methylation states of DNA that can be used in the methods and kits of the invention is combined bisulfite restriction analysis (COBRA). COBRA is a quantitative technique described by Xiong et al. for determining DNA methylation levels at specific genetic loci in small amounts of genomic DNA (Nucleic Acids Res 1997, 25: 2532-2534), which is hereby incorporated by reference. The technique uses restriction enzyme digestion to reveal methylation-dependent sequence differences in PCR products of sodium bisulfite-treated DNA. Methylation levels in an original DNA sample are represented by the relative amounts of digested and undigested PCR product. This ratio has been shown to be linearly quantitative over a broad range of DNA methylation levels. The method has also been applied to DNA sample harvested from microdissected paraffin-embedded tissue samples, thus facilitating the analysis of tissue from a subject by eliminating the need for immediate processing of such samples.

### Detectable Labels

In some embodiments, nucleic acids used in methods of the invention may be detectably labeled. The term “detectable label” as used here means a molecule preferably selected from, but not limited to, fluorescent, enzyme, radioactive, metallic, biotin, chemiluminescent, and bioluminescent molecules. A wide variety of detectable labels are available for use in methods of the invention and may include labels that provide direct detection (e.g., fluorescence, colorimetric, or optical, etc.) or indirect detection (e.g., enzyme-generated luminescence, epitope tag such as the FLAG epitope, enzyme tag such as horseradish peroxidase, labeled antibody, etc.). A non-limiting example of use of a detectable label in a method of the invention is the incorporation of a fluorescent or radioactive label in an amplification reaction (e.g., in PCR). Other methods that may use detectable labels include, but are not limited to methylation assays such as the MethyLight assay, etc. A variety of methods may be used to detect a detectable label depending on the nature of the label and other assay components. Labels may be directly detected through optical or electron density, radioactive emissions, nonradiative energy transfers, etc. or indirectly detected with antibody conjugates, strepavidin-biotin conjugates, etc. Methods for using and detecting labels are well known to those of ordinary skill in the art.

### Differential Expression and Microarray Analysis

Methods of the invention relate, in part, to assessment of abnormal methylation amounts and patterns of genes of the invention. As described herein, abnormal methylation amounts and/or patterns in genes may result in differential expression of the abnormally methylated gene. Methods of the invention include, in addition to methods of assessing DNA to determine methylation levels, may also include additional methods of assaying for differential gene expression of one or more genes of the invention. Differentially expressed genes may be indicated by expression products (i.e., mRNA and/or proteins/polypeptides) that are differentially expressed in a sample as compared to a control. Abnormal methylation patterns in the DNA of a cell may result in differential expression of that DNA. A gene that is differentially expressed or has a differential level of methylation in a sample compared to a control may be referred to herein as a cancer-associated gene. A level of methylation of one or more cancer-associated genes of the invention can be at least about 1%, 5%, 10%, 20%, 30%, 40%, 50%, 60%, 70%, 80%, 90%, 100%, 200%, 300%, 400%, 500%, 600%, 700%, 800%, 900%, 1000%, 2000%, 3000%, 4000%, 5000%, 10,000%, or more higher (including all percentages in between the listed percentages) than a control level of methylation of the one or more genes of the invention.

Differentially expressed genes can be identified in a number of ways. If the expression product is a nucleic acid (i.e., an mRNA), then the differentially expressed gene may be identified using techniques such as subtractive hybridization, differential display, representational difference analysis, or reverse transcriptase-quantitative PCR(RT-qPCR). An approach aimed at identifying differentially expressed transcripts may include conversion of RNA to at least a first strand cDNA.

Another technique that is useful for identifying differentially expressed transcripts involves DNA chip technology and cDNA microarray hybridization. Standard and custom-made DNA chips are commercially available from manufacturers such as Affymetrix and InCyte. High-throughput screening for difference expressed genes and sequences is readily accomplished (Von Stein, et al., Nucleic Acids Res, 1997, 25:2598-602; Carulli, et al., J Cell Biochem Suppl 1998, 30-31: 286-96). These and other methods of assaying for differential gene expression may be in methods of the invention to assess methylation of one or more genes of the invention.

The invention, in some aspects, may also include methods of detecting expression products of genes of the invention. Such methods may be used to identify expression products of genes of the invention, e.g., proteins/polypeptides encoded by a gene of the invention. Such methods may be used for a number of purposes, including, but not limited to confirming the identity of one or more genes (e.g., as tumor suppressor genes), confirmation of a diagnosis, etc. of cancer and/or precancerous condition in cells and subjects, etc. Methods of the invention for detecting expression products of genes of the invention may include, but are not limited to ELISA, immunohistochemistry, immunofluorescence, Western blotting, etc. Those of skill in the art will be able to use such methods and/or alternative methods for the detection and assessment of expression products of one or more genes of the invention.

### General Methods

A level of methylation of one or more genes of the invention can be determined in a number of ways when carrying out the various methods of the invention. In one particularly important measurement, a level of methylation of one or more genomic sequences is measured in relation to a level of unmethylated genomic sequences. Thus, the measurement may be a relative measure, which can be expressed, for example, as a percentage of total positions for methylation in one or more genes of the invention, or as a percentage of the overall methylation level in set or panel of one or more genes of the invention. Those of ordinary skill in the art will appreciate that relative amounts of methylated and unmethylated genomic sequences may be determined by measuring either the relative amount of methylated genomic sequence or the relative amount of unmethylated genomic sequence. In other words, if 90% of an genomic sequence of the invention in an individual cell is unmethylated, then 10% of the genomic sequence in the individual cell is methylated. Thus, measuring the level of methylated genomic sequence may be carried out using a method that measures the relative amount of unmethylated genomic sequence.

Another measurement of the level of methylation of one or more genes of the invention may be a measurement of absolute level methylation of one or more genes of the invention. This could be expressed, for example, in methylation levels per unit of cells or tissue, or as fraction of methylated target sites/total target sites. As used herein, the term “target site” means a CpG dinucleotide. Another measurement of the level of methylation of one or more genes of the invention may be a measurement of the change in the level of methylation of the one or more genes of the invention over time. This may be expressed in an absolute amount or may be expressed in terms of a percentage increase or decrease over time.

Methods of the invention for assessing levels of methylation of genomic sequences of the invention are useful to characterize methylation levels by monitoring changes in the absolute or relative amounts of methylation levels in a subject or sample (e.g., a cell culture) over time. For example, it is expected that an increase in methylation of one or more genes of the invention in a cell or tissue correlates with the presence and/or severity of cancer in the cell or tissue. Accordingly one can monitor levels of methylation of one or more genes of the invention over time to determine if the status of a subject's cancer or cancer cells are changing. Changes (e.g., an increase) in relative or absolute methylation levels of one or more genes of the invention that are greater than 0.1% may indicate an increasing abnormality. Preferably, the change in a level of methylation of the one or more genes of the invention that indicates an abnormality, is greater than 0.2%, greater than 0.5%, greater than 1.0%, 2.0%, 3.0%, 4.0%, 5.0%, 7.0%, 10%, 15%, 20%, 25%, 30%, 40%, 50%, or more. Increases in amounts of methylation of one or more genes of the invention over time may indicate a change in cancer or precancerous condition status in a sample or subject.

Importantly, levels of methylation of one or more genes of the invention can be determined using methods of the invention and are advantageously compared to controls according to the invention. The control may be a predetermined value, which can take a variety of forms. It can be a single cut-off value, such as a median or mean. It can be established based upon comparative groups, such as in groups having normal amounts of methylation of the genomic sequence of the invention and groups having abnormal amounts of methylation of one or more genes of the invention. Another example of comparative groups may be groups having cancer or a precancerous condition, groups that have symptoms of cancer or a precancerous condition, groups without cancer or a precancerous condition, and groups without symptoms of cancer or of a precancerous condition. Another comparative group may be a group with a family history of cancer or a precancerous condition and a group without such a family history. A predetermined value can be arranged, for example, where a tested population is divided equally (or unequally) into groups, such as a low-risk group, a medium-risk group and a high-risk group or into quadrants or quintiles, the lowest quadrant or quintile being individuals with the lowest risk and lowest amounts of methylation of a genomic sequence of the invention and the highest quadrant or quintile being individuals with the highest risk and highest amounts of methylation of a genomic sequence of the invention.

The predetermined value, of course, will depend upon the particular population selected. For example, an apparently healthy population will have a different ‘normal’ range than will a population that is known to have a cancer or a precancerous condition. Accordingly, the predetermined value selected may take into account the category in which an individual or cell falls. Appropriate ranges and categories can be selected with no more than routine experimentation by those of ordinary skill in the art. As used herein, “abnormal” means not normal as compared to a control. By abnormally high it is meant high relative to a selected control. Typically the control will be based on apparently healthy normal individuals in an appropriate age bracket or apparently healthy cells.

It will also be understood that controls according to the invention may be, in addition to predetermined values, samples of materials tested in parallel with the experimental materials. Examples include samples from control populations or control samples generated through manufacture to be tested in parallel with the experimental samples.

One aspect of the present invention relates to the use of the methods of the invention for detecting levels of methylated genomic sequences in an in vitro (e.g., histological or cytological specimens, biopsies, fluid samples, and the like), and, in particular, to distinguish the level of methylation of one or more genes of the invention in the sample from the level of methylation of the one or more genes of the invention in a control sample or a subject. This method involves utilizing a suitable method to assay the methylation state of one or more genes of the invention in a cell and or tissue sample. Methods of the invention may be used for a variety of diagnostic and experimental methods, including, but not limited to (1) diagnosis of cancer and precancerous conditions; (2) determining onset, progression, and/or regression of cancer or a cancerous condition in a subject; (3) characterizing the impact of the level of methylated gene sequences on cancer or a precancerous condition in a subject; (4) evaluating a treatment for altering the level of methylation of a gene of the invention in a subject; (5) evaluating treatments for cancer or a precancerous condition; (6) selecting a treatment for cancer or a precancerous condition in a subject; and (7) monitoring efficacy of a treatment for cancer or for a precancerous condition. Thus, subjects can be characterized, treatment regimens can be monitored, treatments can be selected and disease status can be better understood using the assays of the present invention.

### Diagnostics

Methods of the invention are useful in one aspect in methods for measuring the level of methylation of one or more genes of the invention as a diagnosis of cancer or a precancerous condition. The impact of the level of methylation of one or more genes of the invention on a cell or subject thus can be measured due to the positive correlation between the level of methylation of the sequence and the stage of cancer or a precancerous condition in the cell or subject. The level of methylation in a gene of the invention thus may correlate with the status of cancer or a precancerous condition in a subject. A relatively high level of methylation of a one or more genes of the invention may reflect a more severe type of cancer and/or a more advanced stage of cancer or a precancerous condition in a subject and a lower level of methylation (although still above normal levels, e.g., levels in a cancer-free subject) may reflect a less severe type of cancer and/or a less advanced stage of cancer or a precancerous condition in a subject.

As used herein, the term “diagnose” means the initial recognition of cancer or a precancerous condition in a cell, tissue, and/or subject and also may mean determination of the status or stage of cancer or a precancerous condition in the cell, tissue, and/or subject. For example, a diagnosis of cancer or a precancerous condition in a subject using a methods of the invention may include the determination of the stage of cancer, and/or pathogenic features of cancer in the subject. Thus, the level of expression and/or the level of methylation of one or more genes of the invention can be used to determine the stage or status of cancer in a subject. High levels of methylation of one or more genes of the invention in a sample from a subject may be correlated with advanced stage cancer, with concomitant advanced pathologic features in the cells and tissues of the subject. Similarly, a lower level of methylation of one or more genes of the invention in a sample from a subject may be correlated with a less advanced stage cancer, (e.g. early presymptomatic and late presymptomatic stages) or precancerous with concomitant less advanced pathologic features in the cells and tissues of the subject. Thus, the relative levels and changes in the level of methylation and/or expression of one or more genes of the invention provide diagnostic information about the stage and status of cancer in a cell, tissue, and/or subject.

It will be understood by those of ordinary skill in the art that the level of methylation of some cancer-associated genes may be higher in cancer and some may be lower in cancer when compared to a control. Thus, determination of the level of methylation of a set of cancer-associated genes in a sample may include some cancer-associated genes with higher methylation levels and some with lower methylation levels than that found in a control sample. Some cancer-associated genes may be methylated at an early presymptomatic or precancerous stage at a level that is higher than a normal methylation level (e.g., a control level) and the methylation level may decrease (or increase) at a later presymptomatic and/or symptomatic state. Some cancer-associated genes may be methylated or their expression repressed when the subject is asymptotic for the cancer. It will be understood that the level of methylation of some cancer-associated genes may be higher at more progressive stages of cancer, including metastasis. Due to methylation levels, expression of some cancer-associated genes of the invention may increase and some may decrease at different stages of cancer, (e.g., high methylation may result in low levels of expression and low or no methylation levels may result in higher levels of expression). Thus, in some embodiments, determination of a methylation pattern of more than one cancer-associated gene may be used as an indication of the stage or status of cancer in a cell, tissue, or subject.

The diagnosis of cancer is not limited to analysis of the methylation pattern of one or more genes and may be combined with diagnosis methods routine in the art. These diagnostic assays include but are not limited to histopathology, immunohistochemistry, flow cytometry, cytology, patho-physiological assays, including MRI and tomography, and biochemical assays. Biochemical assays include but are not limited to mutation analysis, chromosomal analysis, ELISA analysis of specific proteins, platelet count etc. Those of ordinary skill in the art will be aware of numerous diagnostic and staging protocols and parameters that are routinely utilized in the art.

Methods and/or kits of the invention can be used to screen patients for diseases associated with the presence of abnormal levels methylation (increased or decreased levels versus a control level) of one or more genes of the invention in which an abnormal level of methylation is associated with cancer or a precancerous condition. As used herein, the term “increased” means higher, for example higher versus a control level. Methods of the invention may be used to diagnose the status and/or stage of cancer or a precancerous condition by assessing the level of methylation in one or more genes of the invention in a sample from a subject or culture of cells that have cancer or a precancerous condition.

The invention, in some aspects, includes various assays to determine levels of methylation of one or more genes of the invention for which abnormal methylation (e.g., hypermethylation) is associated with cancer. Methods and assays of the invention (e.g., methylation assays, examples of which are provided herein) may be used to monitor changes in methylation state of genes in a cell sample and or a subject over time. Thus, methods of the invention may be used to examine changes in specific methylation patterns of one or more genomic sequences (e.g., genes) of the invention, in a subject or cell sample (e.g., cell culture) over time. This allows monitoring of methylation levels in a subject who is believed to be at risk of developing cancer or a precancerous condition and also enables quantitative monitoring in a subject who is known to have cancer or a precancerous condition. Methods of the invention also permit monitoring of a cell or subject for residual disease (e.g., minimal residual cancer or a precancerous condition) and permit monitoring of a cell or subject's response to treatment of cancer or treatment of a precancerous condition. Thus, methods of the invention may be used to diagnose or assess cancer or a precancerous condition in a subject.

### Onset, Progression, Regression

Methods and/or kits of the invention can be used to obtain useful prognostic information by providing an early indicator of disease onset, progression, and/or regression. The invention includes methods to monitor the onset, progression, or regression of cancer in a subject by, for example, obtaining samples at sequential times from a subject and assaying such samples for the level of methylation of one or more genes. A subject may be suspected of having cancer or may be believed not to have cancer and in the latter case, the sample may serve as a normal baseline level for comparison with subsequent samples.

Onset of a condition is the initiation of the changes associated with the condition in a subject. Such changes may be evidenced by physiological symptoms, or may be clinically asymptomatic. For example, the onset of cancer may be followed by a period during which there may be cancer-associated pathogenic changes in the subject, even though clinical symptoms may not be evident at that time. The progression of a condition follows onset and is the advancement of the pathogenic (e.g. physiological) elements of the condition, which may or may not be marked by an increase in clinical symptoms. Onset of a cancer condition may be indicated by a change in the level of methylation of one or more genes of the invention in samples obtained form the subject. For example, if the level of methylation of one or more genes of the invention is lower in a first sample from a subject, than in a second or subsequent sample from the subject, it may indicate the onset or progression of cancer. In contrast, the regression of a condition may include a decrease in physiological characteristics of the condition, perhaps with a parallel reduction in symptoms, and may result from a treatment or may be a natural reversal in the condition.

Progression and regression of cancer may be generally indicated by the increase or decrease, respectively, of the level of methylation of one or more genes of the invention in a subject's samples over time. For example, if the level of methylation of one or more genes of the invention is low in a first sample from a subject and increased levels of methylation are determined to be present in a second or subsequent sample from the subject, it may indicate the progression of cancer and/or additional cancer pathogenesis. Regression of cancer and/or a reduction in pathogenesis may be indicated by finding that the level of methylation of one or more genes of the invention in a sample from a subject are decreased in a second or subsequent sample from the subject relative to the level in a first sample obtained from a subject at an earlier time. Methods of the invention can also be used to detect the presence of minimal residual disease.

### Assays for Efficacy of Treatment

Methods of the invention may also be used to assess the efficacy of a therapeutic treatment of cancer or a precancerous condition and for assessing the level of methylation of one or more genes of the invention in a subject at various time points. For example, a level of a subject's methylation of one or more genes of the invention can be obtained prior to the start of a therapeutic regimen (either prophylactic or as a treatment of cancer or a precancerous condition), during the treatment regimen, and/or after a treatment regimen, thus providing information on the effectiveness of the regimen in the subject. Methods of the invention may be used to compare levels of methylation of one or more genes of the invention in two or more samples obtained from a subject at different times. In some embodiments, a sample is obtained from a subject, the subject is administered a treatment for cancer or a precancerous condition and a subsequence sample is obtained from the subject. A comparison of a subject's levels of methylation of one or more genes of the invention measured in samples obtained at different times and/or on different days provides a measure of the status of the subject's cancer or precancerous condition and can be used to determine the effectiveness of any treatment for cancer or a precancerous condition in a subject.

As used herein, the term “treatment” encompasses both prophylactic and therapeutic treatment, and it embraces the prevention of cancer and precancerous conditions, and the inhibition and/or amelioration of pre-existing cancers and precancerous conditions. A subject receive treatment because the subject has been determined to be at risk of developing cancer or a precancerous condition, or alternatively, the subject may have such a disorder. Thus, a treatment may reduce or eliminate cancer altogether or prevent it from becoming worse. “Evaluation of treatment” as used herein, means the comparison of a subject's levels of methylation of one or more genes of the invention is measured in samples obtained from the subject at different sample times, preferably at least one day apart. In some embodiments, the time to obtain the second sample from the subject is at least 5, 10, 20, 30, 40, 50, minutes after obtaining the first sample from the subject. In certain embodiments, the time to obtain the second sample from the subject is at least 1, 2, 3, 4, 5, 6, 7, 8, 9, 10, 11, 12, 13, 14, 15, 16, 17, 18, 19, 20, 21, 22, 23, 24, 36, 48, 72, 96, 120 or more hours after obtaining the first sample from the subject.

### Treatment

As used herein, a treatment may be a surgical treatment, a chemotherapy treatment, a radiation treatment, etc. Cancer treatment methods are known in the art and the invention embraces all cancer treatment methods. Non-limiting examples of cancer treatment are chemotherapy, radiation and surgical therapy. Non-limiting examples of chemotherapy are monoclonal antibody therapy, the administration of DNA topoisomerase inhibitors, DNA synthesis inhibitors (like cisplatin); or inhibitors of cell surface receptors (like Gleevec® (immatinib mesylate)) and cytokine therapy. In some embodiments, a cancer treatment may include administration of an agent or compound that reduces methylation of one or more genomic sequences of the invention. Thus, in some embodiments of the invention, a treatment strategy may include administration of a methylation inhibitor to a cell or subject to reduce methylation of one or more genes of the invention that are hypermethylated in cancer or in a precancerous condition. Non-limiting examples of DNA methylation inhibitors include 5-azacytidine, 5-aza-2′deoxycytidine (also known as Decitabine in Europe), 5,6-dihydro-5-azacytidine, 5,6-dihydro-5-aza-2′deoxycytidine, 5-fluorocytidine, 5-fluoro-2′deoxycytidine, and short oligonucleotides containing 5-aza-2′deoxycytosine, 5,6-dihydro-5-aza-2′deoxycytosine, and 5-fluoro-2′deoxycytosine. Those of ordinary skill in the art will recognize that similar assessments of candidate therapeutics can be tested in vitro by assessing any change in methylation levels of one or more genomic sequences of the invention that occur in response to contact of the cell with a candidate agent for treatment of cancer or a precancerous condition.

### Selecting Treatment

In some embodiments, methods of the invention may be used to help select a treatment for a subject with cancer or a precancerous condition. Selection of a treatment for a cancer or precancerous condition may be based upon selecting subjects who have abnormally high levels of methylation of one or more genomic sequences of the invention or may be based on a pattern of hypermethylation of one or more genes of the invention. Methods of selecting a treatment may be useful to assess and/or adjust treatment of subjects already receiving a drug or therapy (e.g., radiation treatment or surgery) for treating cancer or a precancerous condition. Based on the determination of the methylation state of one or more genes of the invention, it may be appropriate to alter a therapeutic regimen for a subject. For example, detection of an increase in methylation of one or more genes of the invention in a subject who has received or is receiving a cancer or precancerous-condition treatment may indicate that the treatment regimen should be adjusted (e.g., the dose or frequency of dosing, increased, new treatment initiated, etc.). In some embodiments, a subject may be free of any present treatment for cancer and monitoring of methylation levels according to methods of the invention may identify the subject as a candidate for a treatment for cancer or a precancerous condition, (e.g., treatment to decrease the methylation level of one or more genes of the invention). Thus, subjects may be selected and treated with elevated levels of the same drugs or with different therapies as a result of assays of methylation states of genes of the invention.

According to the present invention, some subjects may be free of symptoms otherwise calling for treatment with a particular therapy, and determining the level of methylation of to one or more genes of the invention may identify the subject as needing treatment. This means that absent the use of the methods of the invention to assess levels of methylation of one or more genes of the invention, the subject would not according to convention as of the date of the filing of the present application have symptoms calling for treatment with a particular cancer therapy or a therapy for a precancerous condition. As a result of measuring the level of methylation of one or more genes of the invention and finding elevation in methylation of one or more genes of the invention, the subject become a candidate for treatment with the therapy.

### Screening for Candidate Therapeutic Agents

The invention also embraces methods for screening for candidate therapeutic agents or candidate treatments to prevent and/or treat cancer and/or precancerous conditions. Assessment of efficacy of candidate therapeutic agents and strategies may be done using assays of the invention in subjects (e.g., non-human animals) and in cells from culture. A candidate therapeutic agent is defined as a compound or molecule that can change the methylation level of one or more of the genomic sequences of the invention. A candidate treatment may be a surgical treatment, radiation treatment, etc.). In some embodiments administration to the subject of an agent, or exposing a sample to the candidate agent or candidate treatment, will result in an increase of the level of methylation of one or more genomic sequences of the invention. In other embodiments administration to the subject of a candidate agent or treatment, or exposing a sample to the candidate agent or treatment, will result in a decrease of the level of methylation of one or more genomic sequences of the invention.

In one embodiment of the invention a sample comprising cancer cells is exposed to a candidate therapeutic agent or treatment. A sample comprising similar cancer cells, or cancer cells of the same lineage and passage, as the cancer cells exposed to the candidate, that have not been exposed to the agent or treatment will function as a control. The level of methylation of one or more cancer-associated genes is monitored upon administration of the candidate therapeutic or treatment, and the change in level of methylation is compared to the change in level of methylation of the one or more genes in the control. If the level of methylation of one or more genes of the invention in the sample that has been exposed to the agent has changed compared with the control sample, the agent is a candidate therapeutic or candidate treatment. In some embodiments the level of methylation of one or more genes of the invention in the sample exposed to the agent or treatment is lower than the level of methylation of one or more genes of the invention in the control.

Methods of screening for agents or treatments that modulate levels of methylation of one or more genes of the invention are encompassed by the invention. Screening methods may include mixing the candidate agent with cells or tissues or in a subject or exposing cells or tissues or a subject to the candidate treatment and using methods of assaying methylation state of one or more gene of the invention to determine the level of methylation before and after contact with the candidate agent or treatment. A decrease in the amount of methylation of a gene of the invention compared to a control is indicative that the candidate agent or treatment is capable of treating cancer or a precancerous condition in a cell, tissue, and/or subject.

In some embodiments an assay mixture for testing a candidate agent comprises a candidate agent. A candidate agent may be an antibody, a small organic compound, or a polypeptide, and accordingly can be selected from combinatorial antibody libraries, combinatorial protein libraries, or small organic molecule libraries. Typically, pluralities of reaction mixtures are run in parallel with different agent concentrations to obtain a different response to the various concentrations. Typically, one of these concentrations serves as a negative control, i.e., at zero concentration of agent or at a concentration of agent below the limits of assay detection.

Any molecule or compound can be a candidate therapeutic. Non-limiting examples of candidate therapeutics are small molecules, RNA including siRNAs, DNA including aptamers, and proteins including antibodies and antibody fragments. The invention also embraces candidate therapeutic with different modes of action. Non-limiting examples of modes of action of candidate therapeutics are methylation inhibitors, DNA modifying agents and agents that bind and hybridize to DNA.

Candidate agents encompass numerous chemical classes, although typically they are organic compounds, proteins or antibodies (and fragments thereof that bind antigen). In some preferred embodiments, the candidate agents are small organic compounds, i.e., those having a molecular weight of more than 50 yet less than about 2500, preferably less than about 1000 and, more preferably, less than about 500. Candidate agents comprise functional chemical groups necessary for structural interactions with polypeptides and/or nucleic acids, and typically include at least an amine, carbonyl, hydroxyl, or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as polypeptides, saccharides, fatty acids, sterols, isoprenoids, purines, pyrimidines, derivatives or structural analogs of the above, or combinations thereof and the like.

Candidate agents are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random or non-random polypeptides, combinatorial libraries of proteins or antibodies, and the like. Alternatively, libraries of natural compounds in the form of bacterial, fungal, plant, and animal extracts are available or readily produced. Additionally, natural and synthetically produced libraries and compounds can be readily be modified through conventional chemical, physical, and biochemical means. Further, known agents may be subjected to directed or random chemical modifications such as acylation, alkylation, esterification, amidification, etc. to produce structural analogs of the agents.

A variety of other reagents also can be included in the mixture. These include reagents such as salts, buffers, neutral proteins (e.g., albumin), detergents, etc., which may be used to facilitate optimal protein-protein and/or protein-agent binding. Such a reagent may also reduce non-specific or background interactions of the reaction components. Other reagents that improve the efficiency of the assay such as protease inhibitors, nuclease inhibitors, antimicrobial agents, and the like may also be used.

The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 0.1 and 10 hours. After incubation, the presence or absence of and/or the level of methylation of one or more genomic sequences of the invention may be detected by any suitable method available to the user. Examples of suitable methods are provided herein and it will be understood by those of ordinary skill in the art that the invention may also encompass the use of additional methods of assaying methyation levels of one or more genomic sequences of the invention.

In some aspects of the invention, kits are provided. Kits of the invention may contain a nucleic acid or other molecule of the invention for use in vitro diagnosis, prognosis, monitoring of cancer or a precancerous condition, and/or testing of candidate cancer or precancerous condition treatments by the methylation assay methods described above. Components of the kits can be packaged either in aqueous medium or in lyophilized form. Reagents for use in methylation assays may also be include in kits of the invention as can detectable labeling agents in the form of intermediates or as separate moieties to be conjugated as part of procedures to assay methylation levels. In some embodiments of a kit of the invention, the kit may include instructions for determining methylation levels and may also include control values (e.g., reference numbers) that can be used for interpreting results of methylation methods used in the invention.

### Kits

Also within the scope of the invention are kits comprising the compositions of the invention and instructions for use. Kits of the invention may be useful for diagnosing cancer or a precancerous condition. Kits of the invention may include a component for determining the level of DNA methylation of one or more cancer-associated genes (e.g., genomic sequences). An example of such a kit may include methods for determining the level of methylation of one, two, or more of the genes of the invention. A kit of the invention may include nucleic acid or protein microarrays nucleic acids of one or more genes of the invention or the polypeptides they encode. Kits may include materials for use in standard techniques of microarray technology to assess expression of one or more genes of the invention.

In some embodiments, a kit of the invention may include PCR components, e.g. primers, solutions, polymerase, etc for amplifying mRNA from a sample or subject. In some embodiments the kit includes components for methylation-specific PCR. Optionally, a kit may include one or more antibodies to one or more polypeptides encoded by the cancer-associated genes along with components useful for use of the antibodies to determine expression of one or more cancer-associated genes in a cell, tissue or subject.

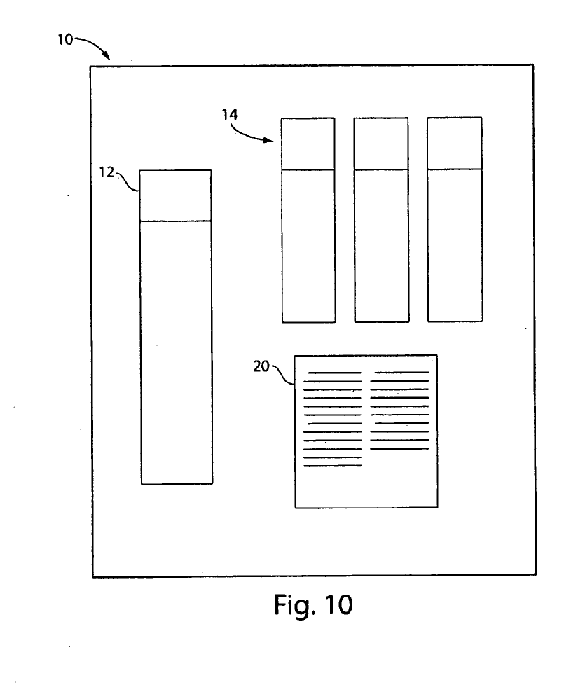

In some embodiments a kit may include components for methylation-inhibitor analysis. These components may include the DNA methylation-inhibitor and means for analyzing differential methylation inhibition, such as a nucleic acid microarray. One embodiment for a kit for diagnosing cancer (10=kit, 12=component for the level of DNA methylation determination; 14=additional components; 20=instructions) is depicted in FIG. 10.

A kit may comprise a carrier being compartmentalized to receive in close confinement therein one or more container means or series of container means such as test tubes, vials, flasks, bottles, syringes, or the like. A first of said container means or series of container means may contain primers for amplifying one or more genomic sequence of the invention or a methylated or unmethylated control sequence. A second container means or series of container means may contain a label or linker-label intermediate for use in a assay of methylation state in a cell, tissue, or subject.

A kit of the invention may also include instructions. Instructions typically will be in written form and will provide guidance for carrying-out the assay embodied by the kit and for making a determination based upon that assay.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

Epigenetic silencing can be reversed by methylation inhibitors. This property of epigenetic silencing has been exploited to perform a microarray based assay to screen for genes that are re-expressed following treatment of melanoma cells with 5AzadC (5-Aza-2′-deoxycytidine). Both DNA methylation and silencing of seventeen genes in melanoma cell lines and in uncultured melanoma tumor samples has been observed. The seventeen genes are PCSK1, BST2, CYP1B1, LXN, SYK, COL1A2, DNAJC15, MFAP2, QPCT, CDH8, LRRC2, CDKN1C, GDF15, HOXB13, DAL1, PTGS2, and WFDC1. DNA methylation has not been previously demonstrated in QPCT, LXN, PCSK1, MFAP2, WFDC1, CDH8 and LRRC2 in any form of cancer.

Several of the above listed genes are candidate tumor suppressors in melanoma. QPCT was methylated in all melanomas and encodes a glutaminyl cyclase that converts precursor glutaminyl peptides to their bioactive pyro-glutaminyl peptide forms (25, 26). LXN was methylated in 95% of melanomas and encodes a global inhibitor of mammalian carboxypeptidases and may function to limit prostate tumor aggressiveness by inhibiting carboxypeptidase 4 (CPA4) (27, 28). COL1A2, which was found to be methylated in 80% of melanomas, has been found to be frequently hypermethylated in several human malignancies including breast cancer, hepatocellular carcinomas, and colorectal cancer (29). Expression of COL1A2 in a tumorigenic cell line led to increased adhesion, slower growth, and reduced colony formation in soft agar, features that are suggestive of a tumor suppressive role for COL1A2 (29).

Two genes involved in the metabolic modification of chemotherapeutic agents were identified with the microarray screen; CYP1B1 which was universally methylated and DNAJC15 was methylated in 50% of melanomas. CYP1B1 is a member of the cytochrome P450 family of mono-oxygenases and has a wide range of substrates, including estrogen, androgens and chemotherapeutic drugs (30). Methylation of CYP1B1 is associated with a poor prognosis in breast cancer (31). DNAJC15 (also known as DNAJD1 and MCJ) is inactivated by promoter hypermethylation in ovarian carcinoma, pediatric brain tumors, and Wilm's tumor (32-34). Loss of DNAJC15 confers resistance to various chemotherapeutic agents used in the treatment of ovarian cancers (32). Thus inactivation of genes involved in metabolic activation of chemotherapeutic drugs may contribute to the drug resistance phenotype commonly observed in malignant melanoma.

The high rates of methylation of CYP1B1 (100%), QPCT (100%), and LAW (95%) in uncultured metastatic melanoma tumor samples suggest that these loci are ideal markers for a variety of melanoma clinical trials. In particular, QPCT could be used as a marker for successful induction of demethylation in melanoma patients being treated with demethylating agents. Alternatively, methylation specific PCR assays for CYP1B1, QPCT, and LXN on blood or serum of melanoma patients could be used as possible staging markers as has recently been described using the lower frequency markers RARB (70%) and RASSF1A (55%)(46). In addition, any gene that is methylated in uncultured metastatic melanoma can be used as a marker for metastatic melanoma.

The SYK cytoplasmic tyrosine kinase plays a role in coupling activated immune receptors to downstream signaling effectors (35). It is expressed in normal breast epithelial tissue and has tumor suppressor properties in breast cancer cells and may affect mitotic progression (36, 37). Promoter hypermethylation of SYK occurs frequently in breast tumors and cell lines and is associated with loss of expression that could be restored upon treatment with 5AzadC (38). Similar findings confirming the role of SYK as a tumor suppressor in melanoma have recently been described (39). The data presented in this study show that promoter hypermethylation is one of the mechanisms that results in loss of SYK expression in melanoma.

HOXB13 is a member of the highly conserved HOX transcription factors that regulate differentiation and pattern formation during embryogenesis (40). HOXB13 has been found to negatively regulate wound healing, possibly by downregulating hyaluronic acid (41, 42). HOXB13 was found to be downregulated in prostate and colorectal cancer cells, where it has an antiproliferative role (43, 44). More recently HOXB13 was found to be epigenetically inactivated in a subset of renal cell carcinomas and had growth inhibitory effects in vitro (45). The data presented in this study show that HOXB13 has tumor suppressive properties in melanoma and is frequently inactivated by promoter region hypermethylation.

### Methods

Tissue specimens, primary cells and cell lines. Melanoma tissue samples were collected in accord with Institutional Review Board-approved protocols at the Memorial Sloan-Kettering Cancer Center and Dana-Farber Cancer Institute. All the cell lines used in the study (MelJuSo, UACC 903, C8161, Neo6/C8161, WM1205 Lu, WM35, Roth, Carney and WM455) were propagated in DMEM-F12 medium (Invitrogen; Carlsbad, Calif.) supplemented with 5% FBS and non-essential amino acids (Invitrogen). Primary cultured human foreskin melanocytes were grown in Medium 254 supplemented with human melanocyte growth serum (Cascade Biologics; Portland, Oreg.). Stable MelJuSo cell lines expressing HOXB13 and SYK were generated by RT-PCR amplification of full-length coding sequence from primary melanocytes and subcloning into the pTRE expression vector (Clontech; Mountain View, Calif.). The inserts were sequenced to ensure an absence of introduced mutations. Stable transfectants were produced by transfection with Lipofectamine (Invitrogen) and selected by growth in media containing G418 (Invitrogen)(800 micrograms/milliliter). Colonies were ring cloned, expanded, and analyzed for transgene expression using RT-QPCR.

5AzadC treatment and microarray analysis. Cells were grown to 50% confluence in 100 mm culture plates and treated with 5 μM 5AzadC (Sigma; St. Louis, Mo.) dissolved in growth media. Fresh 5AzadC media was added every 24 hours until the end of the assay (96 hours). RNA and DNA were isolated from a batch of 5AzadC treated cells every 24 hours starting with 0 hour controls. RNA was isolated following 0 hr and 48 hr 5AzadC treatment of six melanoma cell lines (MelJuSo, UACC 903, c8161, Neo-6 c8161, WM1205 and WM35) and used for the re-expression microarray analysis. Total RNA was isolated using the RNeasy kit (Qiagen; Valencia, Calif.) according to manufacturer's instructions. Preparation of double stranded cDNA and in-vitro transcription (IVT) was as per manufacturer's recommendations. Biotinylated cRNA obtained from IVT were fragmented and hybridized on Human Genome U133A array as per manufacturer's recommendations (Affymetrix; Santa Clara, Calif.). Microarray Suite 5.0 (Affymetrix) software was used to analyze the HG U133A arrays and to determine the statistical significance of a particular probe set. Probe set signal calls with p values greater than 0.01 were not further evaluated. All the arrays were normalized to a target signal intensity value of 500. Baseline analyses were performed using 0 hr treatment array signals as a baseline for the respective 48 hr treatment. The data was further processed using customized programs written for conditional formatting analyses to select for genes whose expression upon 5AzadC treatment was significantly altered in more than one cell line. These genes were examined for presence of CpG islands in their promoter regions using the NCBI mapviewer and the EMBL CpGPlot program (www.ebi.ac.uk/emboss/cpgplot/). Criteria used to identify prospective methylated genes included: 1) upregulation of expression (>4 fold) upon 5AzadC treatment in at least one melanoma cell line; 2) significant expression (MAS 5.0 score of present) in cultured melanocytes, but no significant changes in expression (<2 fold) of the gene upon 5AzadC treatment (in melanocytes); 3) downregulation of expression of the gene in untreated melanoma cell lines compared to primary melanocytes (>4 fold downregulation in at least 2 melanoma cell lines); and 4) presence of a CpG island in the promoter region.

Bisulfite sequencing and quantitative PCR. DNA was isolated from cells and tissues using standard phenol-chloroform extraction. Bisulfite modification was performed as previously described (24) on DNA from melanocytes, melanoma cell lines prior to and after 48 hr treatment with 5AzadC and DNA from melanoma tissues. PCR reactions were carried out using 50 ng of bisulfite modified DNA in a 30 μl volume with a 0.2 μM primer concentration (primer sequences are provided in the Table 2 with the amplified regions of selected genes provided in Table 3). PCR products were purified using the Qiaquick Gel Extraction kit (Qiagen) and directly sequenced on the ABI 3100-Avant automated DNA sequencer. Particular CpG sites were scored as positive if the C:T peak ratio exceeded 1:3 (at least 25% methylation). Real time quantitative PCR was performed for validation of microarray expression data of selected candidate genes by using 2.5 μl of 100 fold diluted cDNA template and 0.2 μM gene specific primers in a 25 μl PCR reaction using JumpStart SYBR green kit (Sigma) according to manufacturer's instructions in an ABI 7700. The reactions were performed in duplicate, CT values obtained were normalized to GAPDH levels and quantification was performed using the comparative CT method.

Western Blotting. Western blotting experiments were performed by separation of 15 μg of cell lysate per sample on SDS-PAGE, transfer to Immunobilon-P membranes (Millipore; Billerica, Mass.), blocking with 0.1M phosphate buffered saline containing 0.2% Tween 20 and 5% non fat milk, and incubation with antisera to HOXB13 (F-9: sc28333), SYK (4D10: sc-1240) (both from Santa Cruz Biotechnology; Santa Cruz, Calif.) or actin (AC-40)(Sigma). Following washes and incubation with peroxidase-conjugated anti-mouse or anti-rabbit secondary antibodies (Amersham Biosciences; Piscataway, N.J.), ECL-Advance detection system (Amersham Biosciences) was employed for chemiluminiscent detection.

In-vitro proliferation assays and tumor formation in nude mice. For in-vitro growth curve assays, three replicates of 10,000 cells each of the vector and HOXB13 or SYK transfected cells were seeded in 6 well cell culture plates for each time point. The cell were trypsinized, diluted and counted on a flow cytometer (Coulter; Fullerton, Calif.) at 24 h intervals starting at 0 h and ending at 120 h. Colony formation assays were performed by plating three replicates each of 1000, 500 or 250 cells of the vector and HOXB13 or SYK transfected cells in 6 well cell culture plates. Colonies were allowed to form for 2 weeks, stained with 0.005% crystal violet. For xenografting experiments, 0.5×10⁶ cells of the vector or HOXB13 or SYK transfected cells were injected subcutaneously into flank skin of nude mice. Four injections were carried out per condition. Tumor dimensions were measured once every week and the mice were sacrificed at 10 weeks post injection when endpoint measurements of tumor weights were obtained. Statistical significance of the endpoint data of the in-vitro and in-vivo assays was evaluated using the Student's paired t-test.

TABLE 1
Genes methylated in uncultured cutaneous malignant melanoma tumors

| | Methylation | |
|---|---|---|
| Gene | incidence | Reference |
| CYP1B1 | 100% | This Study |
| QPCT | 100% | This Study |
| LXN | 95% | This Study |
| COL1A2 | 80% | This Study |
| GDF15 | 75% | This Study |
| RARB | 70% | Guan et al., Int J Cancer 2003, 107: 202 |
| TM | 67% | Furuta et al., Cancer Sci 2004, 95: 962 |
| PCSK1 | 60% | This Study |
| 3-OST-2 | 56% | Takeuchi et al., Oncogene 2006, 25; 7059 |
| RASSF1A | 55% | Hoon et al., Oncogene 2004, 23: 4014 |
| ASC/TMS1 | 50% | Furuta et al., Cancer Res 20o6, 66: 6080 |
| BST2 | 50% | This Study |
| DNAJC15 | 50% | This Study |
| CDKN1C | 35% | This Study |
| MGMT | 34% | Guan et al., Int J Cancer 2003, 107: 202 |
| MFAP2 | 30% | This Study |
| SYK | 30% | This Study |
| MIB1 | 20% | This Study |
| HOXB13 | 20% | This Study |
| PTGS2 | 20% | This Study |
| WFDC1 | 20% | This Study |
| DAPK | 19% | Guan et al., Int J Cancer 2003, 107: 202 |
| APC | 17% | Spugnardi et al., Cancer res 2006, 63: 1639 |
| p16^{INK4A} | 10% | Worm et al., Oncogene 2004, 23: 5211 |
| CDH8 | 10% | This Study |
| p27^{Klp1} | 9% | Worm et al., Oncogene 2004, 23: 5215 |
| PRDX2 | 8% | Herman et al., PNAS 1996, 93: 9821 |
| DAL1 | 5% | This Study |
| LRRC2 | 5% | This Study |

TABLE 2
Bisulfite sequencing primers and PCR conditions.

| | CpG island | | | |
|---|---|---|---|---|
| | sequence | | | |
| | transcript | | | |
| Gene | start = 0 | Primers and Seq ID Nos | Methylation | Accession No. |
| HOXB13 | −110 to +193 | F GAGAGAATAAGTTGGGGTAAAGT | Yes | GeneID: 10481 |
| | | SEQ ID NO: 1 | | UniGene |
| | | R TAACTCCATCCAAAATAACATAAT | | Hs.66731 |
| | | SEQ ID NO: 2 | | |
| LXN | +23 to +220 | F GGATGTAGGGAGTTTGGGTT | Yes | GeneID: 56925 |
| | | SEQ ID NO: 3 | | UniGene |
| | | R TTCCATTGCRAATAAACAATAAC | | Hs.478067 |
| | | SEQ ID NO: 4 | | |
| PTGS2 | +11 to +186 | F GATTTGTAGTGAGYGTFAGGAGTA | Yes | GeneID: 5743 |
| | | SEQ ID NO: 5 | | UniGene |
| | | R CCTATATAACTAAACRCCAAAACC | | Hs.196384 |
| | | SEQ ID NO: 6 | | |
| p57 | +311 to +510 | F GTTYGTGGGATTTTTTTAGTAT | Yes | GeneID: 1028 |
| | | SEQ ID NO: 7 | | UniGene |
| | | R CCACTTCRATCCACTACAAA | | Hs.106070 |
| | | SEQ ID NO: 8 | | |
| SYK | −264 to +62 | F GGTTTATGTTTYGGGTAGTTTTA | Yes | GeneID: 6850 |
| | | SEQ ID NO: 9 | | UniGene |
| | | R CTCCTCCTCRCTCTCCAA | | Hs.371720 |
| | | SEQ ID NO: 10 | | |
| LRRC2 | +109 to +294 | F GGGTAGGTTAGGATTGTTAGTG | Yes | GeneID: 79442 |
| | | SEQ ID NO: 11 | | UniGene |
| | | R CTCCRACAAATTACAAATCAC | | Hs.570630 |
| | | SEQ ID NO: 12 | | |
| PLAB | −113 to +169 | F GTTATTGGAGTGTTTATTTTGTAGGTAG | Yes | GeneID: 9518 |
| | | SEQ ID NO: 13 | | UniGene |
| | | R CAACACCAAAAACATCTAAAAACC | | Hs.616962 |
| | | SEQ ID NO: 14 | | |
| FOS | −225 to −14 | F GTTTAYGAGATTTTTGAGATAGGAA | No | GeneID: 2353 |
| | | SEQ ID NO: 15 | | UniGene |
| | | R CRCAAccACTACTTTTATAACAAAC | | Hs.25647 |
| | | SEQ ID NO: 16 | | |
| QPCT | −48 to +178 | F GGGGTAGGAGGGTTGTAGTTTG | Yes | GeneID: 25797 |
| | | SEQ ID NO: 17 | | UniGene |
| | | R ACCAACAACAACAAATAAAAAATACC | | Hs.79033 |
| | | SEQ ID NO: 18 | | |
| COL1A2 | −75 to +86 | F GGATTGGATAGTTTTTGTTTTGAT | Yes | GeneID: 1278 |
| | | SEQ ID NO:19 | | UniGene |
| | | R CTCTAACTCRTTATCTACATACCTCC | | Hs.489142 |
| | | SEQ ID NO: 20 | | |
| RALGDS | −108 to +163 | F GTTTTITAGTTTGATATTAGTAGGGT | NO | GeneID: 5900 |
| | | SEQ ID NO: 21 | | UniGene |
| | | R TACAACACCACCRAACAACT | | Hs.106185 |
| | | SEQ ID NO: 22 | | |
| BST2 | +24 to +270 | F TATGATTATTGTAGAGTGTTTATGGAAG | Yes | GeneID: 684 |
| | | SEQ ID NO: 23 | | UniGene |
| | | R CTAAACCTCCACATCCTAAAAACC | | Hs.118110 |
| | | SEQ ID NO: 24 | | |
| DUSP6 | −21 to +144 | F GGTTYGGAGAATGTATTTATTGAG | No | GeneID: 1848 |
| | | SEQ ID NO: 25 | | UniGene |
| | | R CCTCCCTCCAAAACTACACCT | | Hs.298654 |
| | | SEQ ID NO: 26 | | |
| WFDC1 | +62 to +342 | F GTGGAATTAAGAAAGTTTAGTAGATTGTG | Yes | GeneID: 58189 |
| | | SEQ ID NO: 27 | | UniGene |
| | | R CTCCCCATCCCAAACACTTAC | | Hs.36688 |
| | | SEQ ID NO: 28 | | |
| CDH8 | −175 to +30 | F TAAGATATTAGTTGTATTTTGGGTTG | Yes | GeneID: 1006 |
| | | SEQ ID NO: 29 | | UniGene |
| | | R AATATCAAACCTCCAAATTCACA | | Hs.368322 |
| | | SEQ ID NO: 30 | | |
| F2RL1 | +75 to +195 | F GGTGCGTTTAGTGGAGTTTTGAG | No | GeneID: 2150 |
| | | SEQ ID NO: 31 | | UniGene |
| | | R ATAACRACCCCCAACAACCAC | | Hs.154299 |
| | | SEQ ID NO: 32 | | |
| MDM2 | −149 to +63 | F TTTATTATTTYGGAGGTGGTG | No | GeneID: 4193 |
| | | SEQ ID NO: 33 | | UniGene |
| | | R CIIACTTCTTACTCCATCTTTCC | | Hs.567303 |
| | | SEQ ID NO: 34 | | |
| MFAP2 | +587 to +854 | F TAGTTGTGAGGAGGAGGAAG | Yes | GeneID: 4237 |
| | | SEQ ID NO: 35 | | UniGene |
| | | R CCTACCTATCCRAATCACTC | | Hs.389137 |
| | | SEQ ID NO: 36 | | |
| RUVBL1 | −148 to +44 | F AGATATAGACGGAAGTGGGTG | No | GeneID: 8607 |
| | | SEQ ID NO: 37 | | UniGene |
| | | R AAAAACCAACAACTAAAACAATAC | | Hs. 272822 |
| | | SEQ ID NO: 38 | | |
| CYP1B1 | −180 to +19 | F GTTTTTATGAAAGTTTGTTGGTAGAG | Yes | GeneID: 1545 |
| | | SEQ ID NO: 39 | | UniGene |
| | | R CCCACTCCCACTTCAAAATC | | Hs.154654 |
| | | SEQ ID NO: 40 | | |
| PCSK1 | −98 to +123 | F GGATTTTAGTTTAGGTAGATTTGA | Yes | GeneID: 5122 |
| | | SEQ ID NO:41 | | UniGene |
| | | R TCCCACCCTCRAACTCTA | | Hs.78977 |
| | | SEQ ID NO: 42 | | |
| DNAJC1S | +307 to +522 | F TGGATTTGYGAGAAGAAAT | Yes | GeneID: 29103 |
| | | SEQ ID NO: 43 | | UniGene |
| | | R TAACAAAACTCACCAATCTCTACT | | Hs.438830 |
| | | SEQ ID NO: 44 | | |
| DAL1 | −165 to +79 | F GGTTGGGAGGGTAGGTAGGA | Yes | GeneID: 23136 |
| | | SEQ ID NO: 45 | | UniGene |
| | | R TTACCTAAAATCAACAAAAAACCC | | Hs.213394 |
| | | SEQ ID NO: 46 | | |
| p21 | −131 to +98 | F GTTGGGTAGTTAGGAGTTTTGGG | No | GeneID: 1026 |
| | | SEQ ID NO: 47 | | UniGene |
| | | R CTCTCTCACCTCCTCTAAATACCTC | | Hs.370771 |
| | | SEQ ID NO: 48 | | |
| EGR1 | −196 to +21 | F TTAGGGTGTAGGATGGAGG | No | GeneID: 1958 |
| | | SEQ ID NO: 49 | | UniGene |
| | | R ACRACTCCCCAAATTCTAC | | Hs.326035 |
| | | SEQ ID NO: 50 | | |

TABLE 3
Exemplary CpG Island sequences

QPCT SEQ ID NO: 51
ggccgcccagccccggggtttgcgaaggtccggggtaggagggctgcagc
ctgcgcgacgcggcgggaggctacccgggggcgatgggaaggcgggcgca
gtcgacccaagggtggagaagagggaaggcgaaggacgcgcgttcccggg
ctcgtgaccgocagcggcccggggaacccgctcccagacagactcggaga
gatggcaggcggaagacaccggcgcgtcgtgggcaccctccacct
LXN SEQ ID NO: 52
agaagcagtcagcccagggctctcggatgcagggagcctgggcccaaaca
gcagcttccggagtcggaaggagctgaggaagaagactgactgaaggagc
ttgcgacttttccgcctcggcaaccggacccagcagcaagcaggacgggc
ggcgctctgctactggtcccgttaagccagagtagcccaagccctgaagt
cactgctcatccggaatgg
PCSK1 SEQ ID NO: 53
ccttccttttcctaatccctggctgcttattttagccgcttctccgcccc
gccgggaataccattcggatcttagtccaggtagatctgacgtcaagaga
tggctttcgtcgatttgacgtgtaaacactcatttccattctggctggga
agggctggggctccactcagcctggagaccgaagcgcttcactgagcgct
cgccgccgcccagcctctcctctcgcgcctcctagctcttcgcagagc
MFAP2 SEQ ID NO:54
ggccaaggggggactgggaatcctggagggccaggtctgggggagagtta
ggaggtcgtgaatttggacttggatgcttgttgaagctgatgcatcttga
ggacatttgtgggacacataggctgggtcagggctgaaagaggtgctggt
tatggccgggggcagggactcatgcctgtaatcccaacagcccaggagga
tgagacaggaggaatgcttgaggccaaaatttcgaggccggaagttccag
acgagcctgggcaacacagcaagaccctgtctctagaaaaggaaagaaag
aaccgctggttgtggaagccagccatggcccagagctcagcagtgtagga
gaggagggtgcgggcctgagagaggcagcaggcttggctggagaggcaga
aggaaaaccaaggcaggagagtgtcctggagctgggagaaggcagaggga
gatcgaggcttactttctggctgaggggcctagggtgagtcactttggga
ggcttgatttcctcctctgtgaaatgggcaacacacctacccttgcccac
ctcacccccctcaccccgtcgacgtcgagtgaggagcagctgtgaggagg
aggaagctttgcacagacgtgaggacggccccgggcgcctctaccccggg
gacaccccccccgccaggagatggaaccccgagggcacggcgtggcggcc
catgcgggcagctcgcgttaggacccagcggctccggggctggagggcgg
gcgccagccccgtcgggggcccggagggggactcggagcgggccaagggg
cggctccggcgggcggactcggagcgggcggcggagtgacccggacaggt
aggcgcgggcctggcgcttgggggccccggggggcggtcaggggcgtcgg
cgggtcccccggcgcgggggcgagggtgccaggctctctgggtcctcgat
cctggggcagccgcggaggttcctgaaccccgcgccttcagagcggaggc
caagggggcacctggaaagtttggggttgctctgcagagagaacgcgaat
tgggaagggcggggagcccgagtttggggggagccagaggtgaggtcgtc
gggggatgggacatcttttgttctcccggggaggttggaatgtggcgggt
ggggtggggggggggggtccggtgggaatggccgagacagagacgcagag
agacaaagagagatcagagacgggagcgcgagggcgcgcggagcaagttt
ccgcggctgattgttcccagctgtgcggcgctgcaacatctgggtttctc
ccgccgggacccctccccctgagcccggctctggagccggcagagccctc
c
WFDC1 SEQ ID NO: 55
gtgctggacgcggacacatgatccgagggaccctgctgggtggaactaag
aaagtccagcagactgtgcacgctcctgtccccactcacaggcccacgca
gcgaggggggcccctcttctgtgtgcgtctggaaggtcgctgcccaggga
ggaaatgcctttaaccggcgtggggccgggcagctgcaggaggcagatca
tccgggctctggcctcttgctacttctcctccacgccggctctgccaaga
atatctggaaacgggcattgcctgcgaggctggccgagaaatcccgtgta
agtgcctgggatggggag
CDH8 SEQ ID NO: 56
cggctcacttccacatcacttcacagttacatttggcaggcaggcgggct
cgcacgccggagcgctcagcccagaattagtggatttatttggaatctcc
ctgcctcctccaagctccgccgccgccgccggcttgtgcatagacggtag
cgtcaagacaccagctgcactttgggttgcggacacccagagcccagcgc
gcccgccctgcgtctccgcaccaagccctgtagctccgagaggcatgaac
ggaatccggaggcgcctgcctagcgagcgaggaccggtcggcgcttgccg
ccctcgggagctaaccccgagcacctccagccatttgtga
LRRC2 SEQ ID NO: 57
atccgcctacaaatatagcctgttagcgcacaggcacgggaggcttcttc
cggctcggccgtgctgctcagcgggtgtaaacagcctctgcggtgtaaac
agcctctgcggtgtaaacagccgcggcgggcaggccaggactgtcagtgc
ccgcccctcggggcaggtaagcgctgggcgtccgaggtggccggggggcg
ggagcgggggccttgcgctccccgtgggaggacgcgggcggatggccggt
gccccgggcgctcagttgtttaatccttcctctgtgccttggtgacctgc
aatctgccggagcgacaggggtaaacgtccctaaccggacagcggctggg
gcttaggcatccaccgttatctgaggtccaggactatggtgggaaggcga
gaaaggagagagcgtggggaggcaggggtccccgcgagtgaatcttgctc
cggcttagcagttcccctgcgcgtactgctccggcgcccaaggctgagcc
caaagctttctggtccaa

### Example 1

### Identification of Candidate Genes Silenced by DNA Methylation in Melanoma

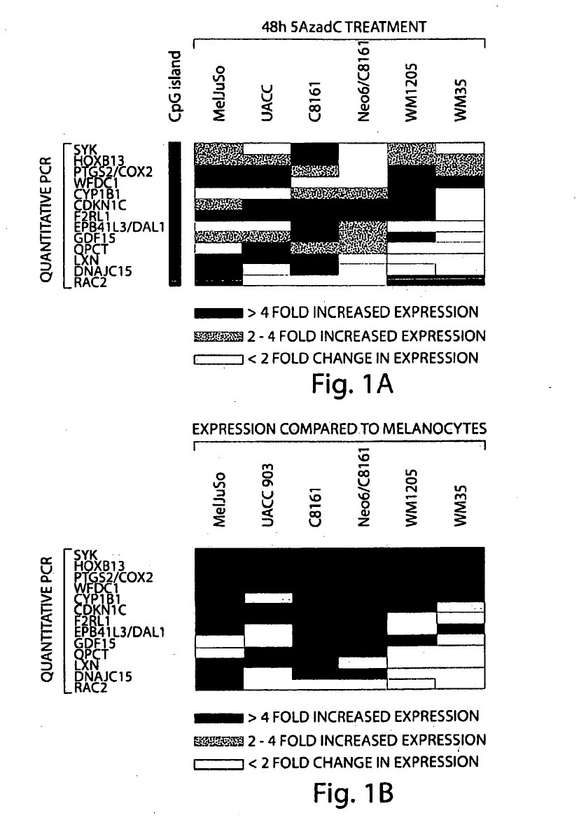

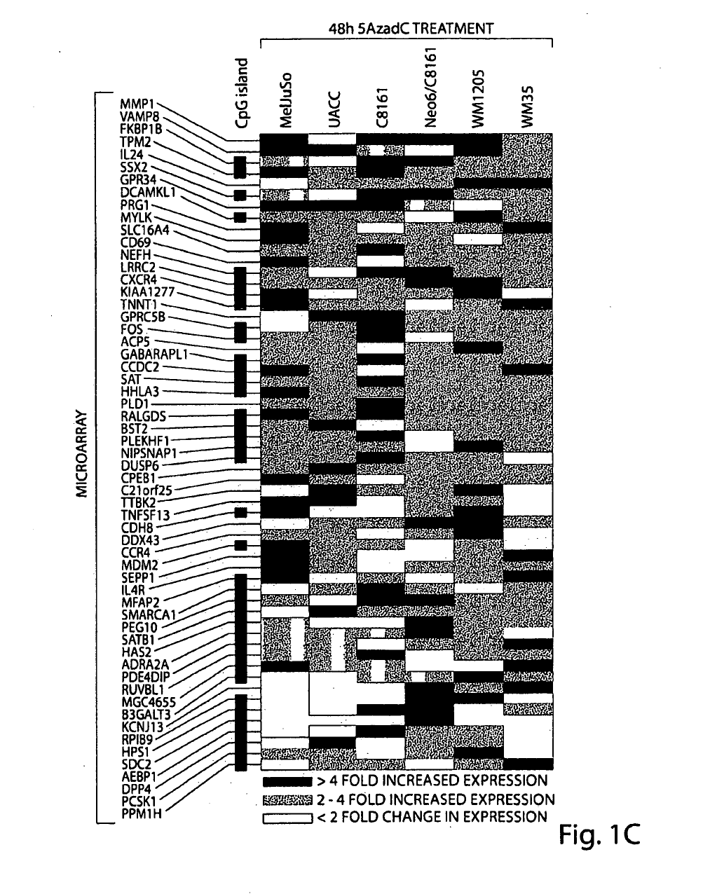

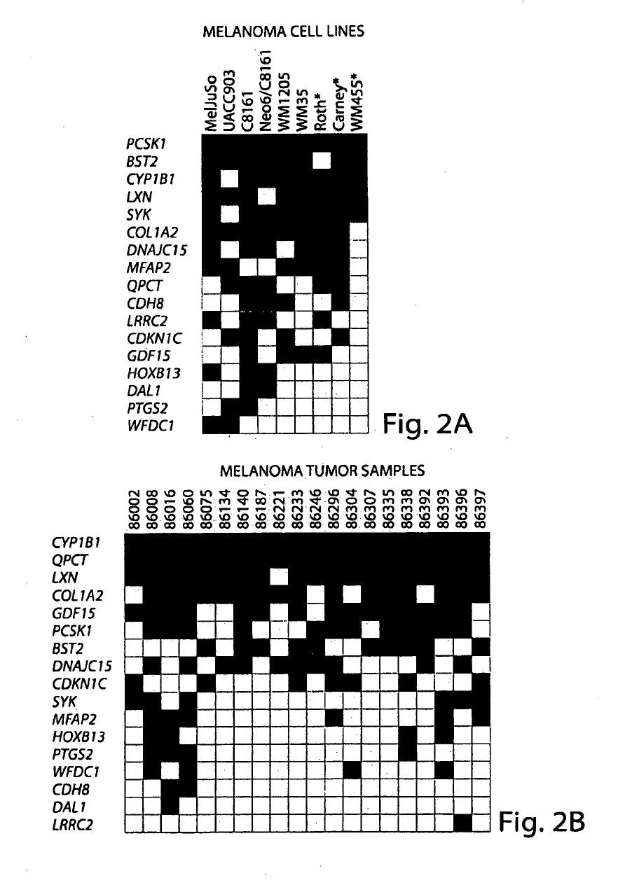

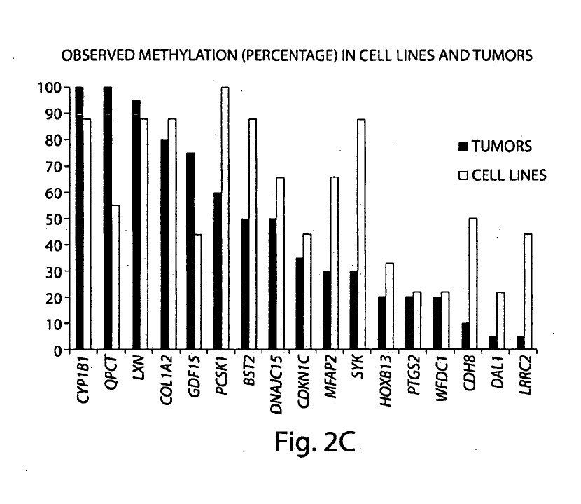

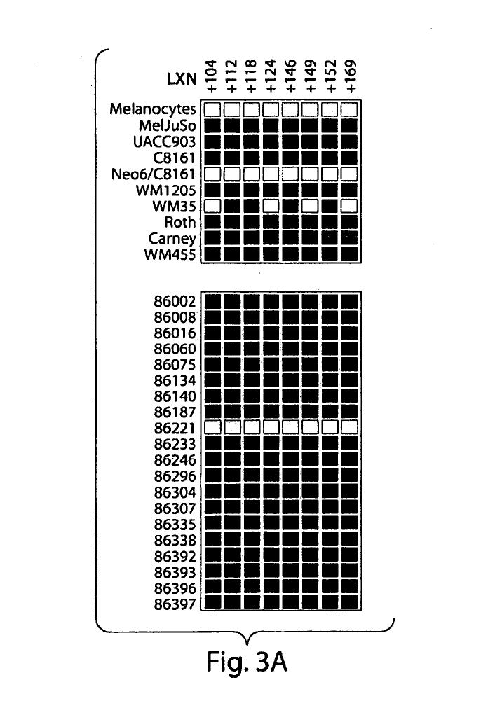

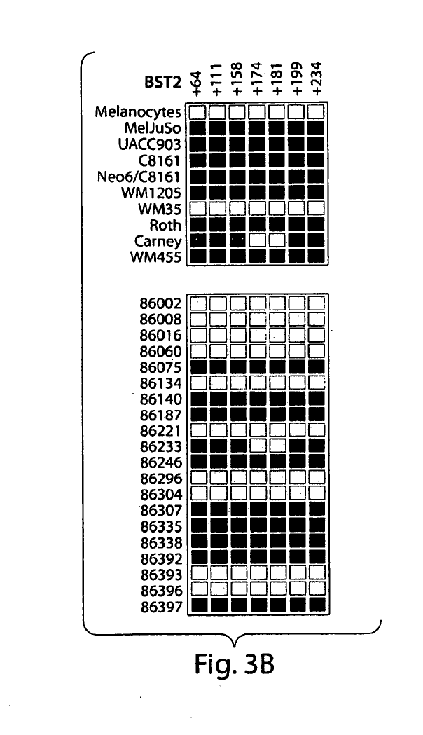

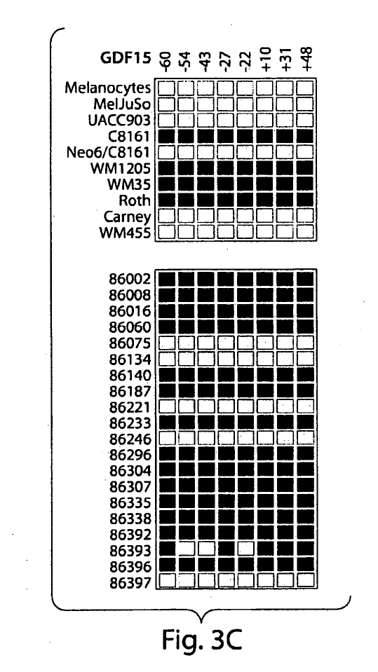

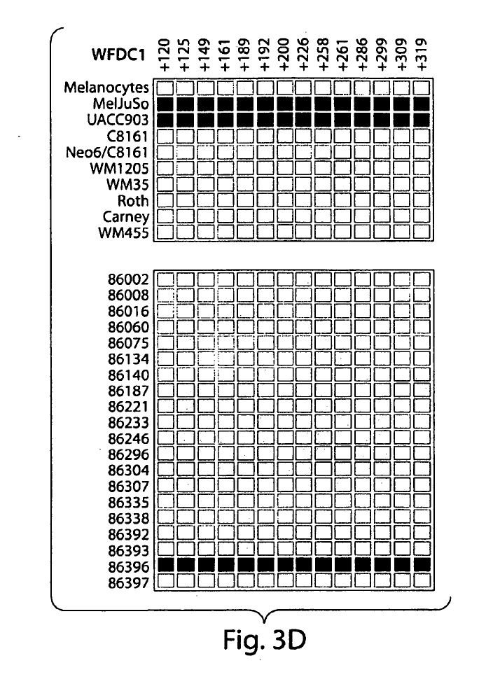

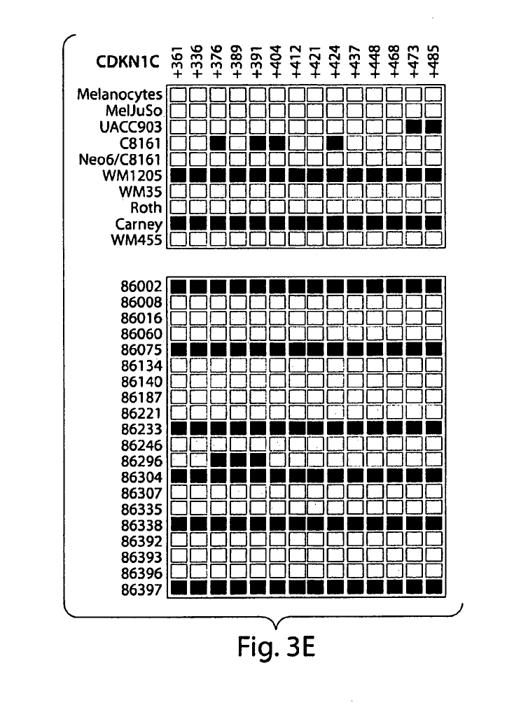

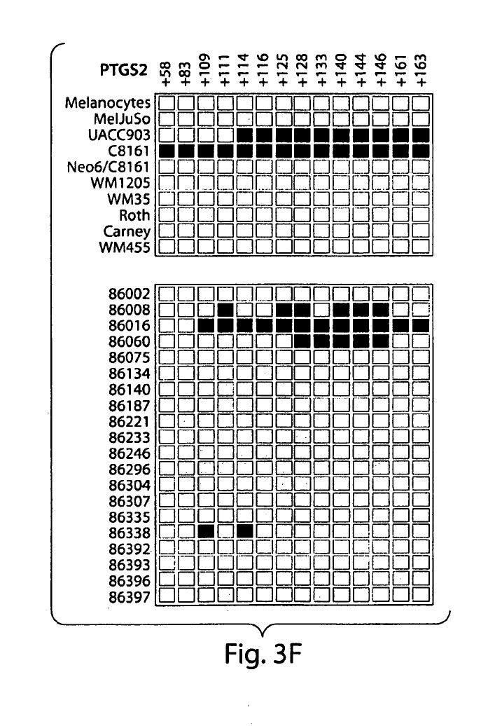

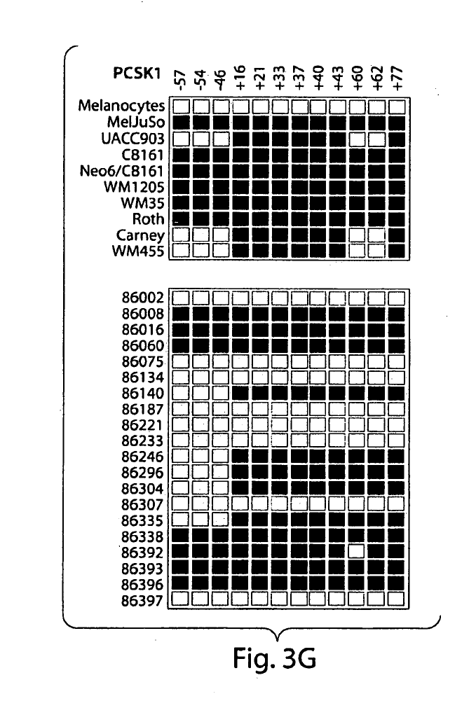

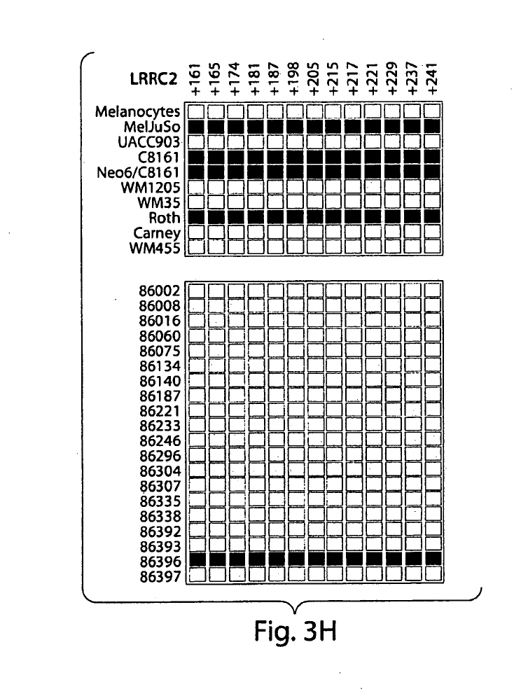

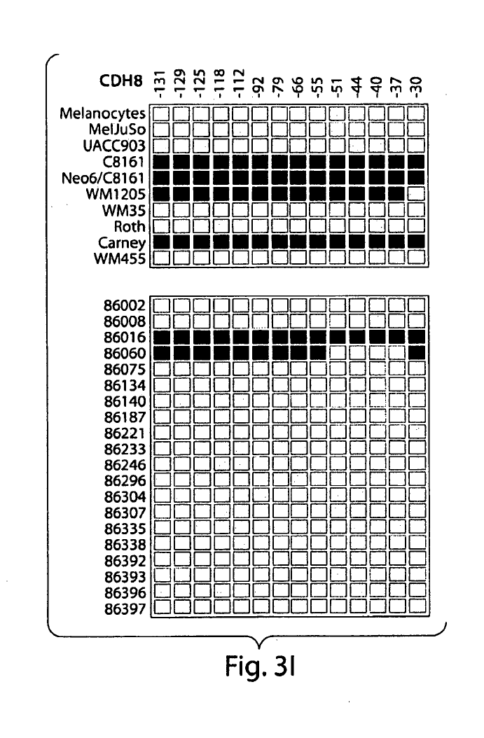

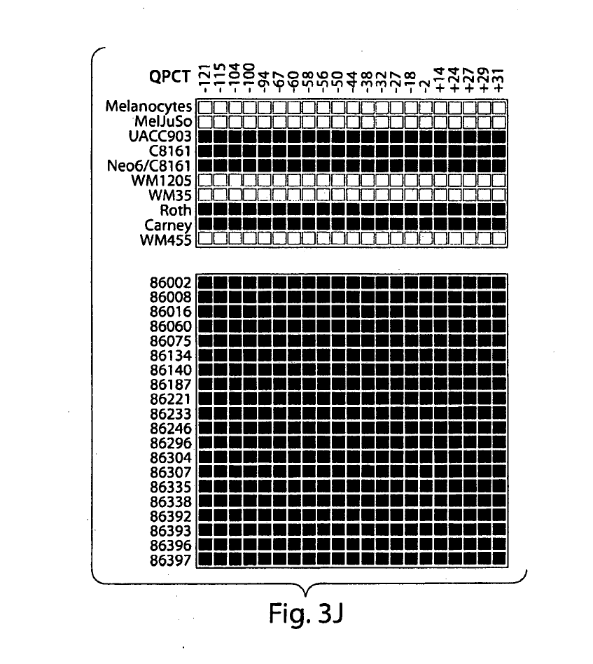

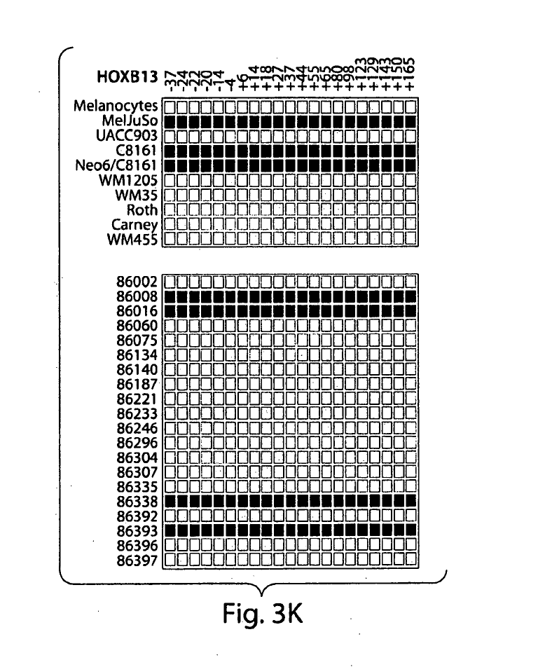

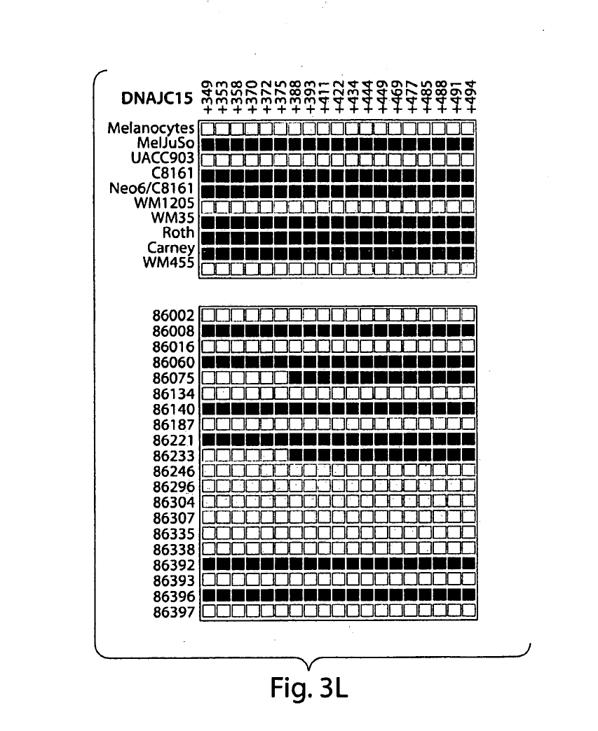

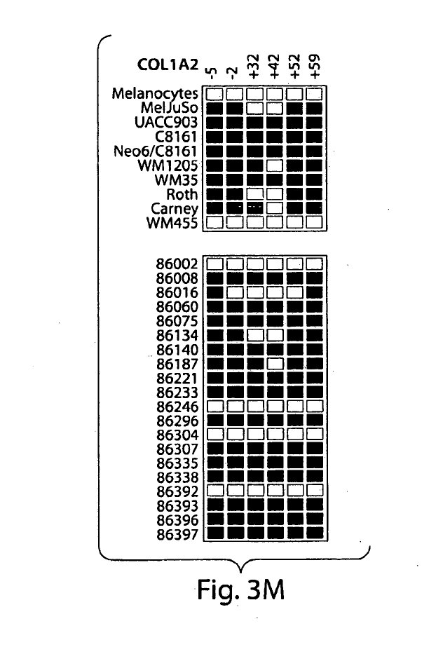

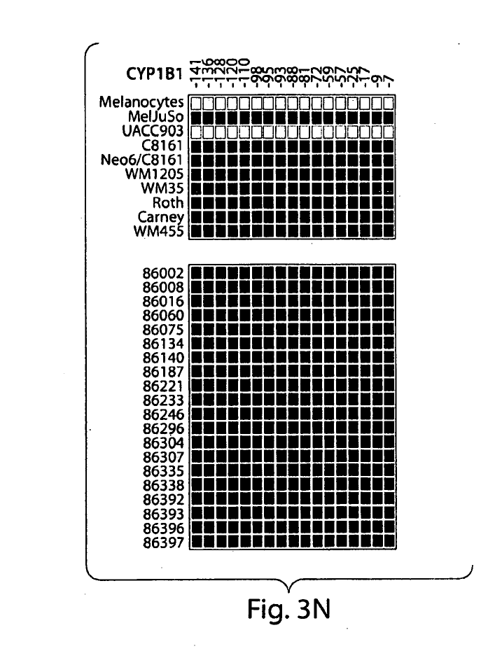

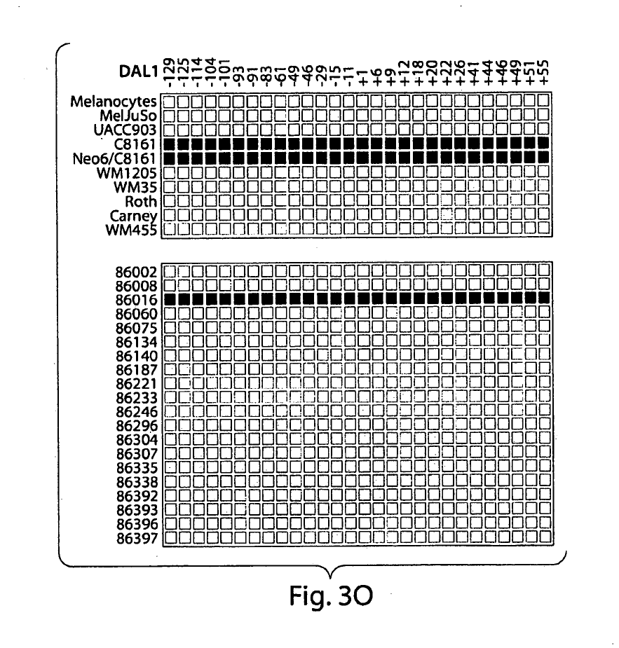

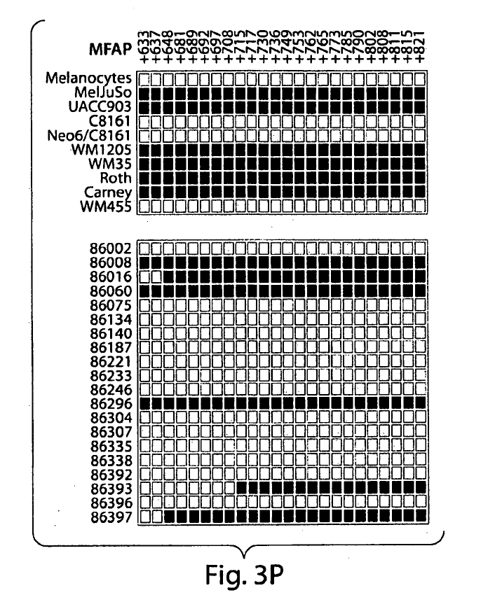

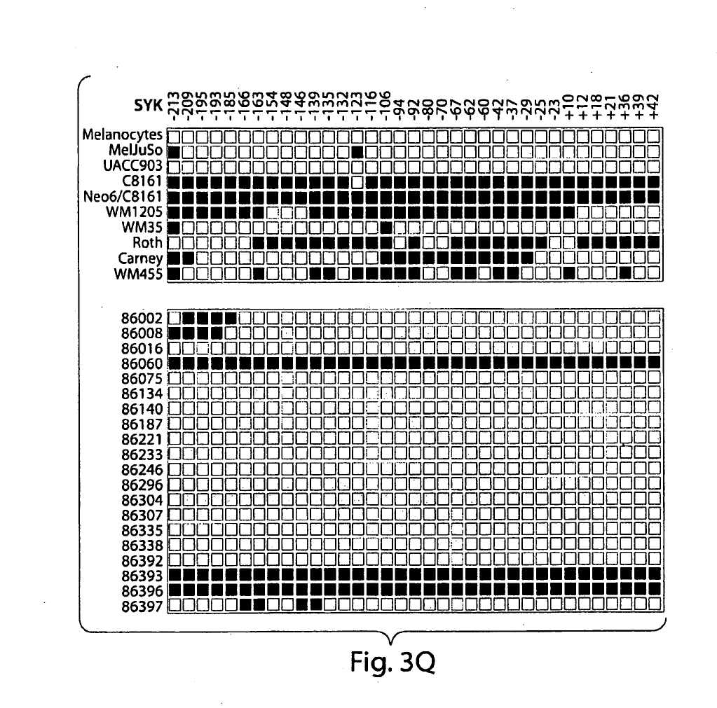

In order to determine DNA methylation-related expression changes, six human melanoma cell lines were treated with 5AzadC and compared for expression changes at 48 hours post-treatment relative to untreated melanoma cell lines. Expression of 150 genes was increased by greater than 4 fold upon 5AzadC treatment in at least one of the six melanoma cell lines. These 150 genes represent a small fraction (0.8%) of the 18,400 transcripts represented on the microarray. Eighty percent (120/150) of the genes with altered expression were associated with promoter region CpG islands, suggesting that these genes might be directly methylated in the melanoma cell lines (FIG. 1). A subset of 25 candidate genes was selected for further validation by bisulfite sequencing using the additional criterion of >4 fold downregulation of expression of the gene in at least 2 untreated melanoma cell lines compared to primary melanocytes. Around two-thirds, 68% (17/25), of these genes were densely methylated in the promoter region in at least one of 9 melanoma cell lines or 20 melanoma tumor tissue samples (FIGS. 2A and B). No DNA methylation was detected in primary human melanocytes at any of the loci. DNA methylation of specific genes occurred at similar frequencies in cell lines and tumor tissues, and was present both in melanoma cell lines and melanoma tissue samples for all genes tested (FIG. 2C). Several of the melanoma cell lines (7/9) and melanoma tumor specimens (7/20) exhibited DNA methylation of greater than 50% of the genes tested. The methylation status of each potential CpG methylation site for all 17 genes in all melanoma cell lines, primary melanocytes, and melanoma tumor samples is depicted in FIG. 3.

### Example 2

### Identification of Methylation-Targeted Tumor Suppressor Candidate Genes in Melanoma

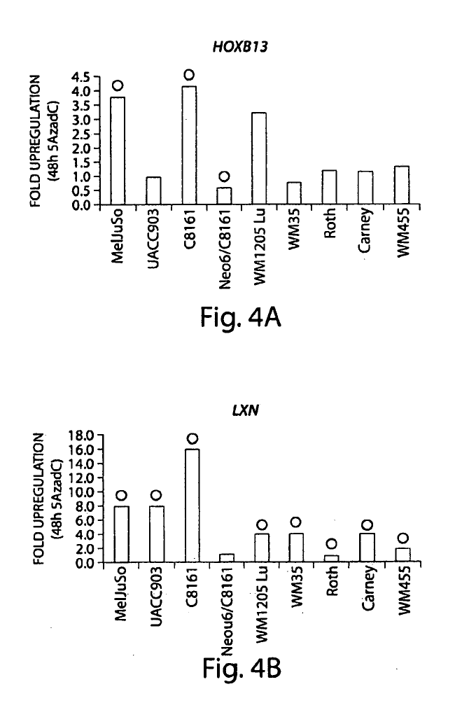

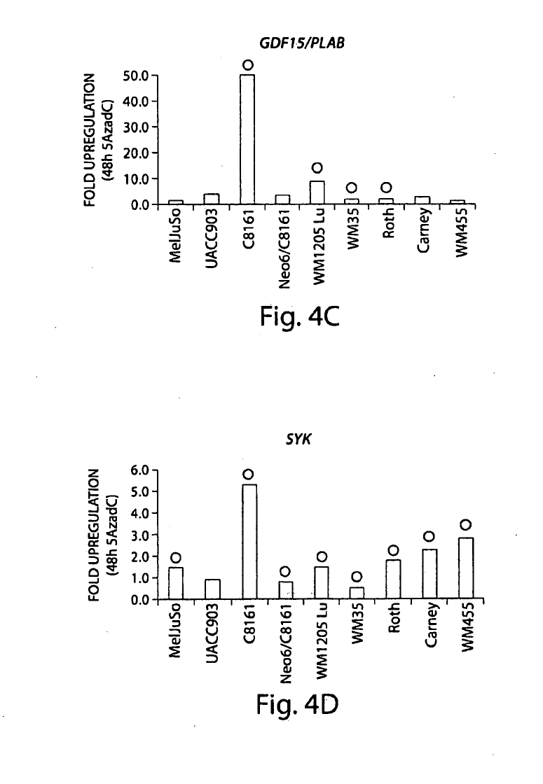

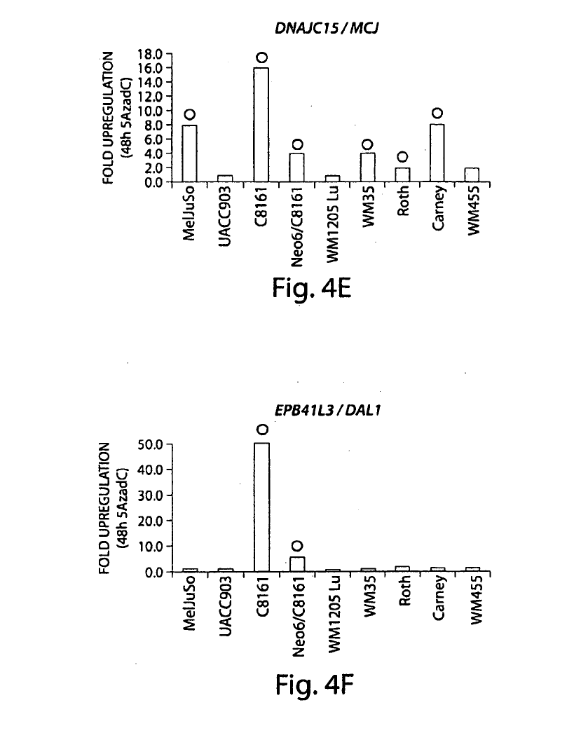

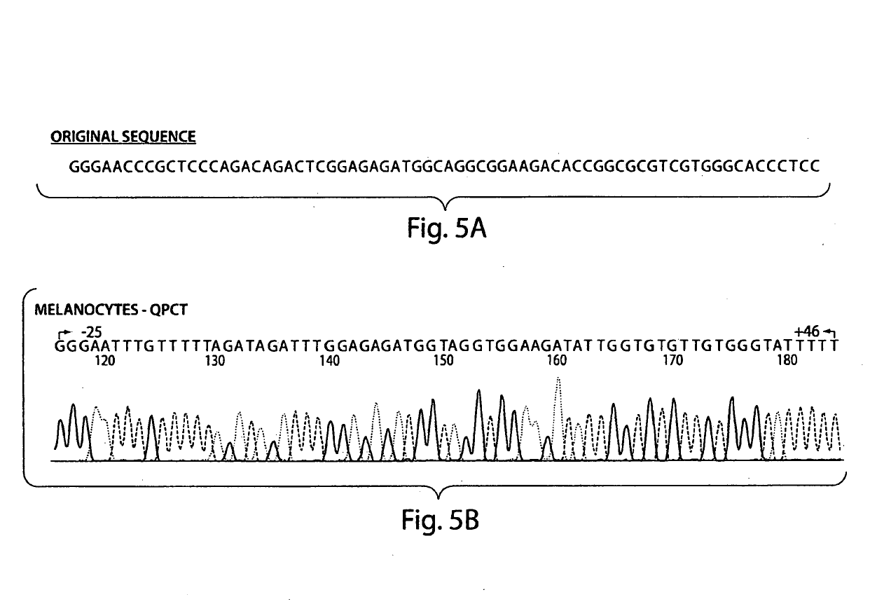

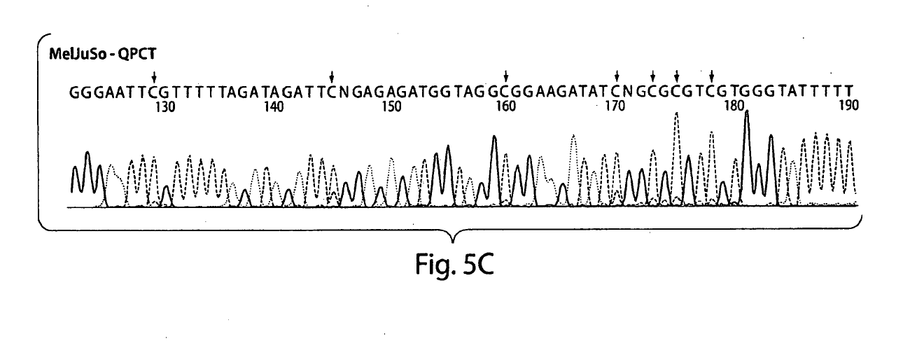

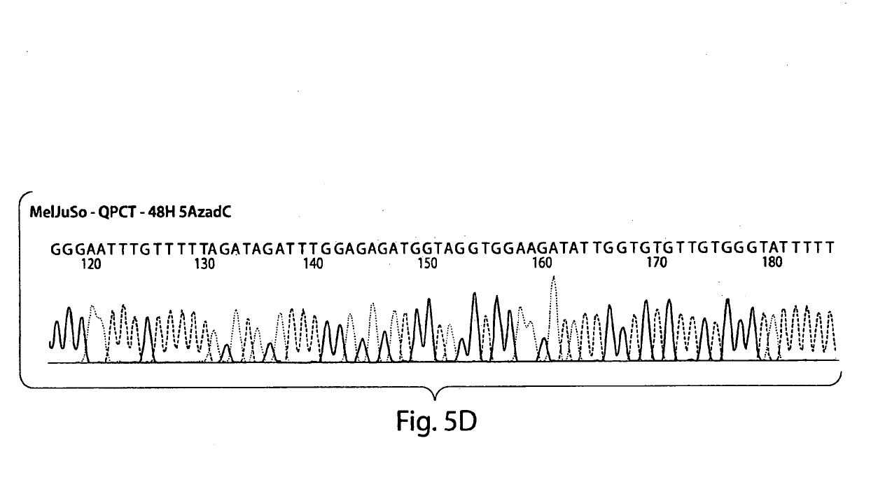

A very high incidence of promoter methylation (>75%) was observed in CYP1B1, QPCT, LXN, COL1A2 and GDF15 and an incidence of 50-60% was observed in PCSK1, BST2 and DNAJC15 in melanoma tumor samples (FIGS. 2B and C; Table 1). The genes CDKN1C, MFAP2, SYK, HOXB13, PTGS2 and WFDC1 were methylated in 20-35% and CDH8, DAL1 and LRRC2 in 5-10% melanoma tumor tissues. Using quantitative RT-PCR, it was confirmed that promoter methylation of several of the above candidate genes was accompanied by reduced expression relative to melanocytes and that this loss of expression could be reversed in the majority of cases by 5AzadC treatment (FIGS. 1A and C and FIG. 4). Demethylation was also induced following 5AzadC treatment (FIG. 5). Two candidate genes, SYK and HOXB13, were evaluated in detail. These genes were selected because of their loss of expression in most melanoma cell lines relative to melanocytes (FIG. 1B).

### Example 3

### Tumor Suppressor Properties of SYK in Malignant Melanoma

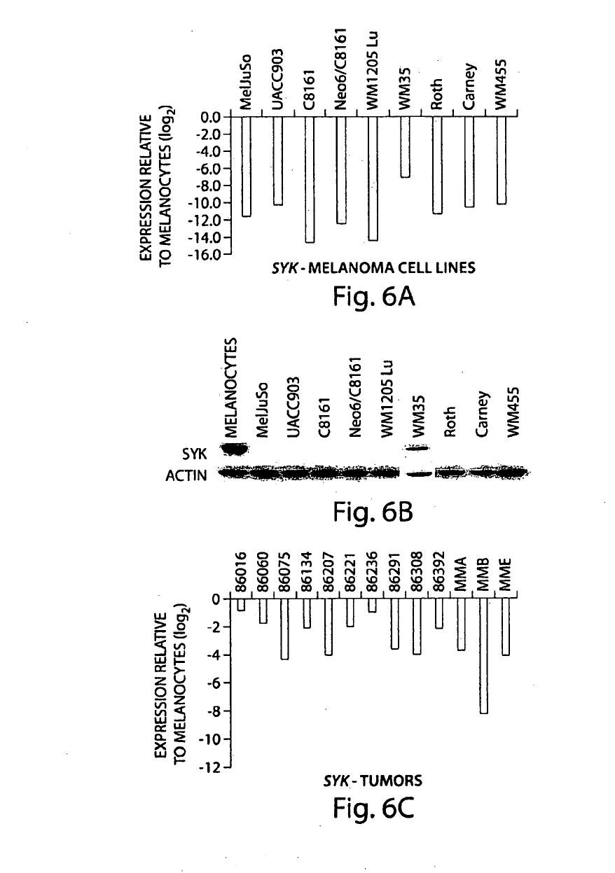

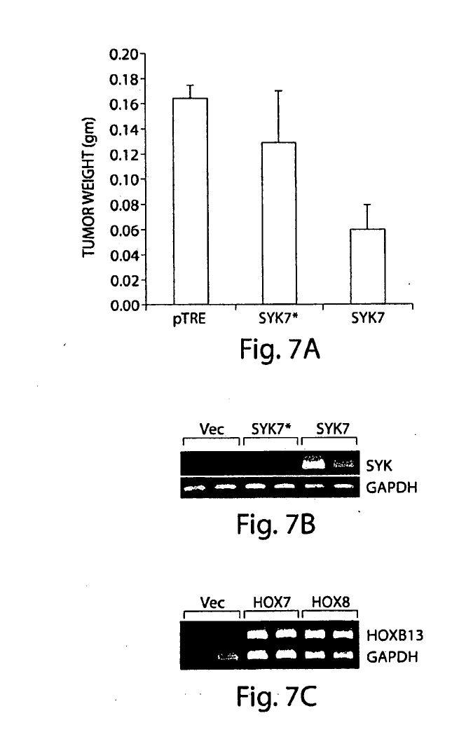

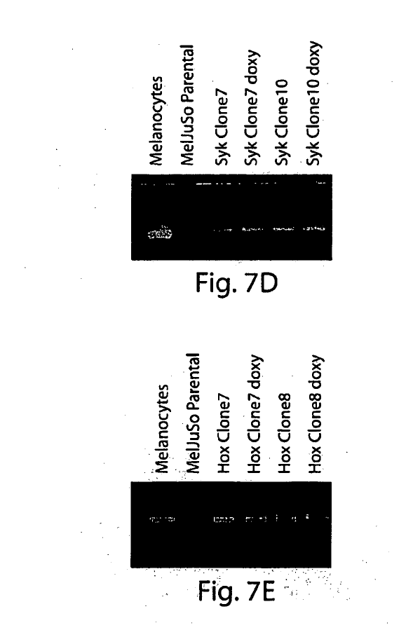

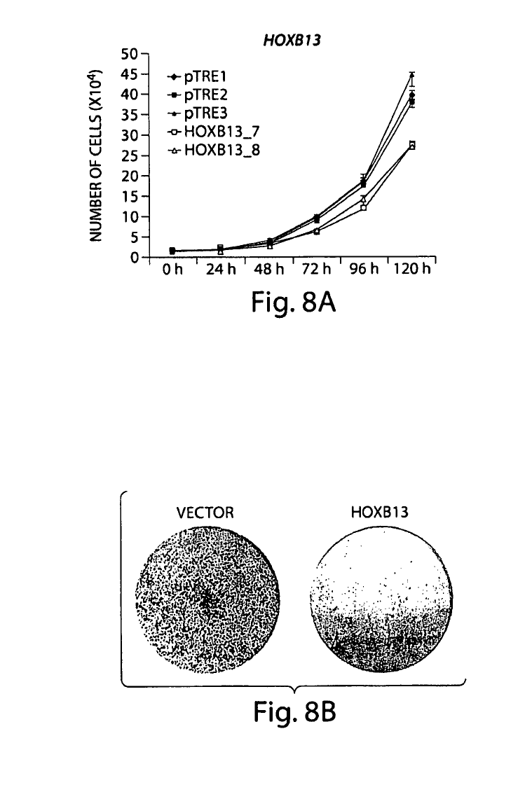

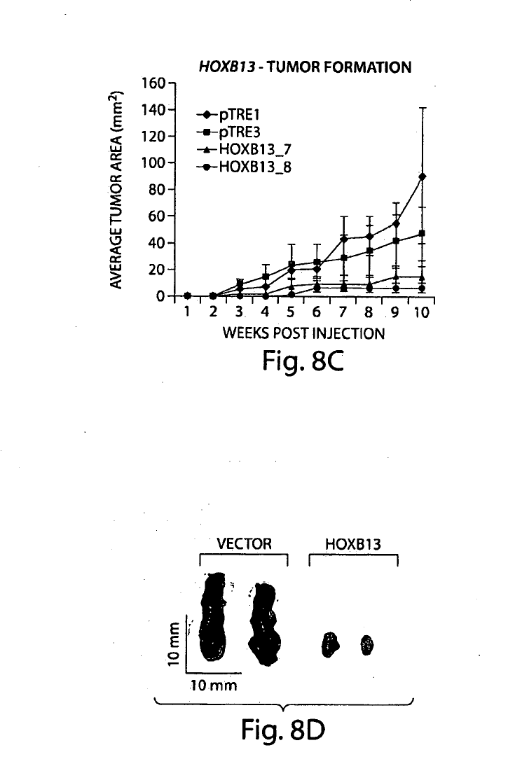

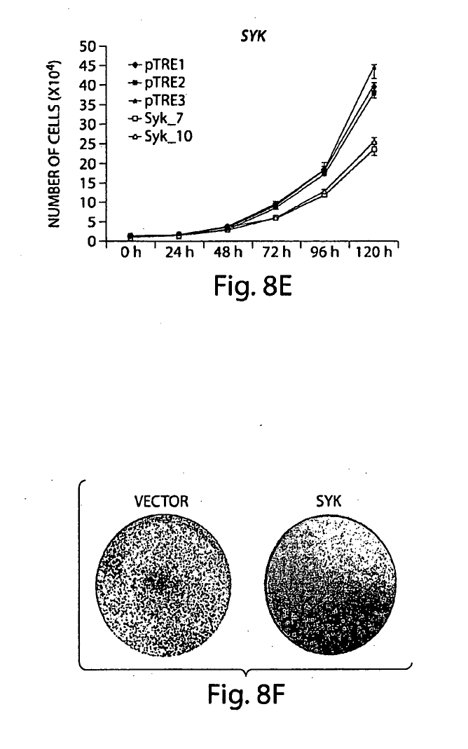

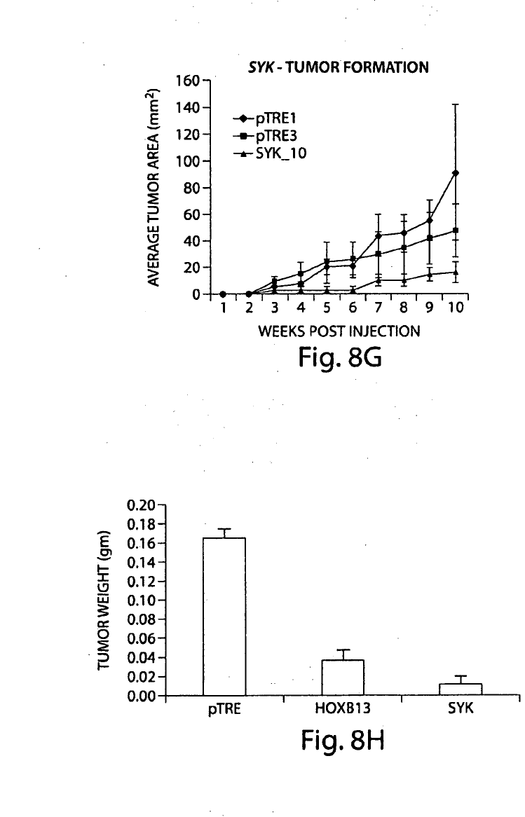

SYK mRNA expression was markedly reduced in all 9 melanoma cell lines compared to primary cultured human melanocytes (FIG. 6A) and 5AzadC treatment resulted in >2 fold upregulation of SYK in a subset (4/8) of cell lines with SYK methylation (FIG. 4B). Immunoblotting showed complete absence of protein expression in all but one cell line (FIG. 6B). SYK mRNA expression was reduced >4 fold in 77% of the primary melanoma tissue samples (10/13) (FIG. 6C). Promoter region methylation of SYK was detected in 89% of the melanoma cell lines and 30% of primary tumors (FIGS. 2A, B, and C). In order to examine if SYK has a tumor suppressor role in melanoma, phenotypic changes resulting from stable re-expression of SYK in a SYK-negative melanoma cell line (MelJuSo) were analyzed. Expression of SYK at comparable levels to those seen in primary melanocytes (FIG. 7) resulted in reduced cell proliferation (FIG. 8B) and markedly reduced colony formation in vitro (FIG. 8B). In subcutaneous xenografting experiments with nude mice, the average tumor size was reduced by greater than eight fold in SYK-expressing clones compared to vector controls (FIG. 8B).

### Example 4

### Tumor Suppressor Properties of HOXB13 in Malignant Melanoma

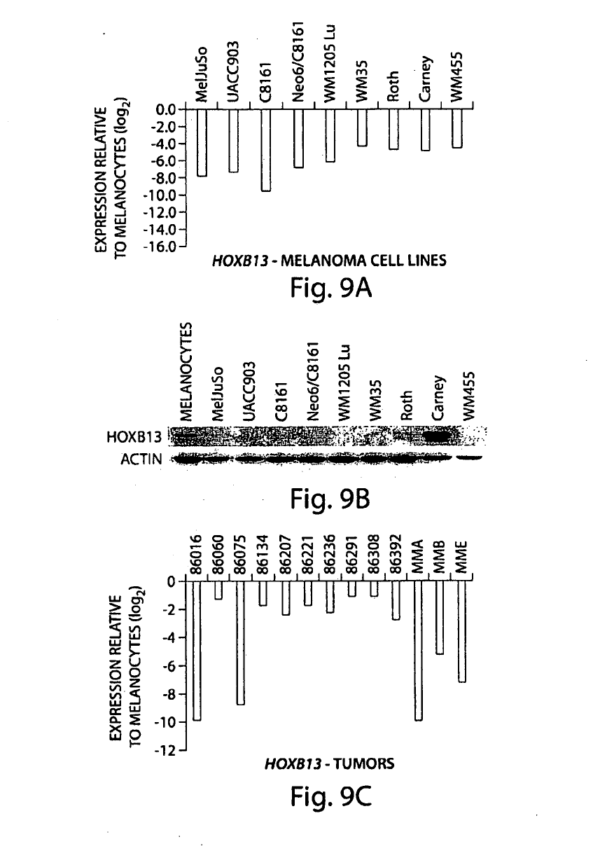

HOXB13 mRNA expression was markedly reduced (>16 fold) in all the melanoma cell lines (FIG. 9A). However, one of the cell lines had retained protein expression (FIG. 9B), which may indicate increased protein stability in that cell line. In the melanoma tumor samples greater than four fold reduction of HOXB13 mRNA was observed, compared to melanocytes in more than 60% (8/13) of the samples (FIG. 9C). Promoter region methylation was detected in 33% of the melanoma cell lines (FIG. 2A) and 20% of the tumor samples (FIG. 2B) tested. Re-expression of HOXB13 occurred upon treatment with 5AzadC in 2 out of 3 cell lines with promoter region methylation (FIG. 4A). HOXB13 was stably expressed in a HOXB13-deficient cell line (MelJuSo) at similar levels to that seen in primary melanocytes (FIG. 7) in order to characterize the possible tumor suppressor function of HOXB13 in melanoma cells. Cell proliferation and colony formation were reduced in HOXB13 transfected clones compared to vector controls (FIG. 8A). HOXB13 transfected MelJuSo lines exhibited greater than 4 fold reduction in tumor size relative to vector controls in xenografting experiments in immunodeficient mice (FIGS. 8A and C). These results indicate that HOXB13 is a tumor suppressor in melanoma and is targeted for silencing by promoter hypermethylation.

### Example 5

### Detection of Methylation in Blood Samples

DNA was prepared from ten samples of blood from individuals using standard methods. The DNA was assessed using bisulfite modification as described above herein. The results indicated that in normal blood samples, e.g. those without known disease (e.g., cancer) lacked detectable LXN methylation as detected by bisulfite modification and DNA sequencing. The results indicated that there is a very low level of background of methylation detected in normal blood. The detection of methylation levels in normal blood samples provides a baseline level of methylation, (e.g., a control) that may be used for testing blood from subjects suspected of having cancer. The results indicated the suitability of the method and assay for use with blood samples for the determination of methylation levels of candidate genes, and for the diagnosis of cancer and/or precancerous conditions.

### Example 6

### Detection of Methylation of Candidate Genes in Blood Samples from Subjects for Diagnosis of Cancer and/or Precancerous Conditions

Blood samples from melanoma patients are examined for the level of LXN methylation as described for DNA assessment herein. The level of LXN methylation is compared between melanoma patient blood samples (e.g., subject sample) and control blood samples (e.g., non-cancer containing samples or a prior sample from a subject undergoing treatment). Similarly to LXN, methylation status of other candidate genes presented in FIG. 3 is examined and compared between melanoma patient blood samples (subject sample) and non-cancer control blood samples. A difference in methylation levels of genes such as LXN and control levels of methylation in a control blood sample, (e.g., hypermethylation in the test sample versus the control level of methylation) indicates that the presence of cancer in the sample. Thus, analysis of methylation of genes such as LXN in blood samples contributes to the diagnosis of cancer. Additional genes that are evaluated include QPCT, PCSK1, MFAP2, WFDC1, CDH8, and LRRC2. Determination of levels of methylation of one or more of the following gene of one or more of the genes, CYP1B1, COL1A2, GDF15, RARB, TM, 3-OST-2, RASSF1A, ACS/TMS1, BST2, DNAJC15, CDKN1c, MGMT, SYK, MiB1, HOXB13, PTGS2, DAPK, APC, p16^{INK4A}, p27^{Kip1}, PRDX2, PYCARD, CDKN2A, CDKN1B, and DAL1 is also performed in blood samples from a subject and from control blood samples. The levels of methylation of candidate genes is compared in the subject sample and control sample to determine whether or not the subject has cancer or a precancerous condition and/or to assess efficacy of a cancer treatment. Assessment of methylation of candidate genes in a blood sample from a subject permits detection and diagnosis of cancer and/or precancerous conditions in the subject.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

The contents of all references, patents and published patent applications cited throughout this application are incorporated herein by reference in their entirety.

### REFERENCES

1. Chin L. The genetics of malignant melanoma: lessons from mouse and man. Nat Rev Cancer 2003; 3:559-70.
2. Kamb A, Shattuck-Eidens D, Eeles R, et al. Analysis of the p16 gene (CDKN2) as a candidate for the chromosome 9p melanoma susceptibility locus. Nat Genet 1994; 8:23-6.
3. Aitken J, Welch J, Duffy D, et al. CDKN2A variants in a population-based sample of Queensland families with melanoma. J Natl Cancer Inst 1999; 91:446-52.
4. Davies H, Bignell G R, Cox C, et al. Mutations of the BRAF gene in human cancer. Nature 2002; 417:949-54.
5. Alsina J, Gorsk D H, Germino F J, et al. Detection of mutations in the mitogen-activated protein kinase pathway in human melanoma. Clin Cancer Res 2003; 9:6419-25.
6. Pollock P M, Harper U L, Hansen K S, et al. High frequency of BRAF mutations in nevi. Nat Genet 2003; 33:19-20.
7. Curtin J A, Fridlyand J, Kageshita T, et al. Distinct sets of genetic alterations in melanoma. N Engl J Med 2005; 353:2135-47.
8. Jones P A and Baylin S B. The fundamental role of epigenetic events in cancer. Nat Rev Genet 2002; 3:415-28.
9. Issa J P. CpG island methylator phenotype in cancer. Nat Rev Cancer 2004; 4:988-93.
10. Bird A. DNA methylation patterns and epigenetic memory. Genes Dev 2002; 16:6-21.
11. Esteller M, Fraga M F, Guo M, et al. DNA methylation patterns in hereditary human cancers mimic sporadic tumorigenesis. Hum Mol Genet 2001; 10:3001-7.
12. Cormier R T and Dove W F. Dnmt1N/+ reduces the net growth rate and multiplicity of intestinal adenomas in C57BL/6-multiple intestinal neoplasia (Min)/+ mice independently of p53 but demonstrates strong synergy with the modifier of Min 1 (AKR) resistance allele. Cancer Res 2000; 60:3965-70.
13. Eads C A, Nickel A E, and Laird P W. Complete genetic suppression of polyp formation and reduction of CpG-island hypermethylation in Apc(Min/+) Dnmt1-hypomorphic Mice. Cancer Res 2002; 62:1296-9.
14. McCabe M T, Low J A, Daignault S, et al. Inhibition of DNA methyltransferase activity prevents tumorigenesis in a mouse model of prostate cancer. Cancer Res 2006; 66:385-92.
15. Yamada Y, Jackson-Grusby L, Linhart H, et al. Opposing effects of DNA hypomethylation on intestinal and liver carcinogenesis. Proc Natl Acad Sci USA 2005; 102:13580-5.
16. Gaudet F, Hodgson J G, Eden A, et al. Induction of tumors in mice by genomic hypomethylation. Science 2003; 300:489-92.
17. Gonzalgo M L, Bender C M, You E H, et al. Low frequency of p16/CDKN2A methylation in sporadic melanoma: comparative approaches for methylation analysis of primary tumors. Cancer Res 1997; 57:5336-47.
18. Worm J, Bartkova J, Kirkin A F, et al. Aberrant p27Kip1 promoter methylation in malignant melanoma. Oncogene 2000; 19:5111-5.
19. Worm J, Christensen C, Gronbaek K, et al. Genetic and epigenetic alterations of the APC gene in malignant melanoma. Oncogene 2004; 23:5215-26.
20. Spugnardi M, Tommasi S, Dammann R, et al. Epigenetic inactivation of RAS association domain family protein 1 (RASSF1A) in malignant cutaneous melanoma. Cancer Res 2003; 63:1639-43.
21. Hoon D S, Spugnardi M, Kuo C, et al. Profiling epigenetic inactivation of tumor suppressor genes in tumors and plasma from cutaneous melanoma patients. Oncogene 2004; 23:4014-22.
22. Guan X, Sagara J, Yokoyama T, et al. ASC/TMS1, a caspase-1 activating adaptor, is downregulated by aberrant methylation in human melanoma. Int J Cancer 2003; 107:202-8.
23. Furuta J, Nobeyama Y, Umebayashi Y, et al. Silencing of Peroxiredoxin 2 and aberrant methylation of 33 CpG islands in putative promoter regions in human malignant melanomas. Cancer Res 2006; 66:6080-6.
24. Herman J G, Graff J R, Myohanen S, et al. Methylation-specific PCR: a novel PCR assay for methylation status of CpG islands. Proc Natl Acad Sci USA 1996; 93:9821-6.
25. Pohl T, Zimmer M, Mugele K, et al. Primary structure and functional expression of a glutaminyl cyclase. Proc Natl Acad Sci USA 1991; 88:10059-63.
26. Fischer W H and Spiess J. Identification of a mammalian glutaminyl cyclase converting glutaminyl into pyroglutamyl peptides. Proc Natl Acad Sci USA 1987; 84:3628-32.
27. Kayashima T, Yamasaki K, Yamada T, et al. The novel imprinted carboxypeptidase A4 gene (CPA4) in the 7q32 imprinting domain. Hum Genet 2003; 112:220-6.
28. Pallares I, Bonet R, Garcia-Castellanos R, et al. Structure of human carboxypeptidase A4 with its endogenous protein inhibitor, latexin. Proc Natl Acad Sci USA 2005; 102:3978-83.
29. Sengupta P K, Smith E M, Kim K, et al. DNA hypermethylation near the transcription start site of collagen alpha2(I) gene occurs in both cancer cell lines and primary colorectal cancers. Cancer Res 2003; 63:1789-97.
30. Murray G I, Melvin W T, Greenlee W F, et al. Regulation, function, and tissue-specific expression of cytochrome P450 CYP1B1. Arum Rev Pharmacol Toxicol 2001; 41:297-316.
31. Widschwendter M, Siegmund K D, Muller H M, et al. Association of breast cancer DNA methylation profiles with hormone receptor status and response to tamoxifen. Cancer Res 2004; 64:3807-13.
32. Shridhar V, Bible K C, Staub J, et al. Loss of expression of a new member of the DNAJ protein family confers resistance to chemotherapeutic agents used in the treatment of ovarian cancer. Cancer Res 2001; 61:4258-65.
33. Ehrlich M, Jiang G, Fiala E, et al. Hypomethylation and hypermethylation of DNA in Wilms tumors. Oncogene 2002; 21:6694-702.
34. Lindsey J C, Lusher M E, Strathdee G, et al. Epigenetic inactivation of MCJ (DNAJD1) in malignant paediatric brain tumours. Int J Cancer 2006; 118:346-52.
35. Sada K, Takano T, Yanagi S, et al. Structure and function of Syk protein-tyrosine kinase. J Biochem (Tokyo) 2001; 130:177-86.
36. Coopman P J, Do M T, Barth M, et al. The Syk tyrosine kinase suppresses malignant growth of human breast cancer cells. Nature 2000; 406:742-7.
37. Zyss D, Montcourrier P, Vidal B, et al. The Syk tyrosine kinase localizes to the centrosomes and negatively affects mitotic progression. Cancer Res 2005; 65:10872-80.
38. Yuan Y, Mendez R, Sahin A, et al. Hypermethylation leads to silencing of the SYK gene in human breast cancer. Cancer Res 2001; 61:5558-61.
39. Hoeller C, Thallinger C, Pratscher B, et al. The non-receptor-associated tyrosine kinase Syk is a regulator of metastatic behavior in human melanoma cells. J Invest Dermatol 2005; 124:1293-9.
40. Krumlauf R Hox genes in vertebrate development. Cell 1994; 78:191-201.
41. Mack J A, Abramson S R, Ben Y, et al. Hoxb13 knockout adult skin exhibits high levels of hyaluronan and enhanced wound healing. Faseb J 2003; 17:1352-4.
42. Brecht M, Mayer U, Schlosser E, et al. Increased hyaluronate synthesis is required for fibroblast detachment and mitosis. Biochem J 1986; 239:445-50.
43. Jung C, Kim R S, Zhang H, et al. HOXB13 is downregulated in colorectal cancer to confer TCF4-mediated transactivation. Br J Cancer 2005; 92:2233-9.
44. Jung C, Kim R S, Lee S J, et al. HOXB13 homeodomain protein suppresses the growth of prostate cancer cells by the negative regulation of T-cell factor 4. Cancer Res 2004; 64:3046-51.
45. Okuda H, Toyota M, Ishida W, et al. Epigenetic inactivation of the candidate tumor suppressor gene HOXB13 in human renal cell carcinoma. Oncogene 2005.
46. Koyanagi K, Mori T, O'Day S J, et al. Association of circulating tumor cells with serum tumor-related methylated DNA in peripheral blood of melanoma patients. Cancer Res 2006; 66:6111-7.
47. Furuta J, Kaneda A, Umebayashi Y, et al. Silencing of the thrombomodulin gene in human malignant melanoma. Melanoma Res 2005; 15:15-20.
48. Furuta J, Umebayashi Y, Miyamoto K, et al. Promoter methylation profiling of 30 genes in human malignant melanoma. Cancer Sci 2004; 95:962-8.
49. Takeuchi T, Adachi Y, Sonobe H, et al. A ubiquitin ligase, skeletrophin, is a negative regulator of melanoma invasion. Oncogene 2006.

What is claimed is:

1. A method for diagnosing cancer or a precancerous condition in a subject, the method comprising:
determining a level of methylation of one or more genes comprising QPCT, LXN, PCSK1, MFAP2, WFDC1, CDH8 and LRRC2 in a sample from a subject,
comparing the level of methylation of the one or more genes in the sample to a control level of methylation of the one or more genes,
wherein a higher level of methylation of the one or more genes in the sample compared to the control level of methylation is diagnostic for cancer or precancerous condition in the subject.

2. (canceled)

3. The method of claim 1, further comprising determining the level of methylation of one or more of genes comprising CYP1B1, COL1A2, GDF15, RARB, TM, 3-OST-2, RASSF1A, ACS/TMS1, BST2, DNAJC15, CDKN1c, MGMT, SYK, MiB1, HOXB13, PTGS2, DAPK, APC, p16^{INK4A}, p27^{Kip1}, PRDX2, PYCARD, CDKN2A, CDKN1B and DAL1.

4. The method of claim 1, wherein the methylation of the one or more genes is located on CpG islands of the one or more genes.

5. The method of claim 1, wherein the methylation of the one or more genes is located on a nucleotide sequence of SEQ ID No. 51-57.

6. The method of claim 1, wherein the cancer is melanoma.

7. (canceled)

8. The method of claim 1, wherein the control level of methylation of the one or more genes is the level of methylation of the one or more genes in a non-cancerous cell.

9. The method of claim 8, wherein the non-cancerous cell is a cultured melanocyte.

10. The method of claim 1, wherein the sample is a fluid sample.

11. (canceled)

12. The method of claim 1, wherein the sample is a tissue sample.

13. (canceled)

14. The method of claim 1, wherein the level of methylation of the one or more genes in the sample is at least 1%, 10%, 20%, 50%, 100%, 200%, 400% or 1000% higher than the control level of methylation of the one or more genes.

15. (canceled)

16. The method of claim 1, wherein the level of methylation of the one or more genes is determined by methylation-specific PCR, methylation-inhibitor analysis, methylation sensitive restriction analysis, sequencing of bisulfite modified DNA, methylation-sensitive single nucleotide primer extension, MethyLight analysis, pyrosequencing, or combined bisulfite restriction analysis.

17-18. (canceled)

19. The method of claim 1 further comprising isolating a nucleic acid from the sample and analyzing the nucleic acid on a nucleic acid microarray.

20. The method of claim 1, further comprising analyzing the sample for one or more mutated genes.

21. (canceled)

22. The method of claim 1, further comprising analyzing the sample for one or more chromosomal instability loci.

23-24. (canceled)

25. A method for determining onset, progression, or regression, of cancer in a subject, the method comprising:
determining a level of methylation of one or more genes comprising QPCT, LXN, PCSK1, MFAP2, WFDC1, CDH8 and LRRC2 in a sample obtained from the subject,
determining the level of methylation of the one or more genes in a second sample obtained from the subject at a later time than the first sample was obtained,
comparing the level of methylation of the one or more genes in the first sample with the level of methylation of the one or more genes in the second sample,
wherein a lower level of methylation of the one or more genes in the first sample compared with the level of methylation of the one or more genes in the second sample indicates onset or progression of cancer in the subject, and
wherein a higher level of methylation of the one or more genes in the first sample compared with the level of methylation of the one or more genes in the second sample indicates regression of cancer in the subject.

26-47. (canceled)

48. The method of claim 25, wherein the subject is undergoing treatment for cancer.

49. The method of claim 25, wherein the subject has been diagnosed with cancer.

50-65. (canceled)

66. A method for selecting a course of treatment of a subject having or suspected of having cancer, the method comprising:
detecting a level of methylation of one or more genes comprising QPCT, LXN, PCSK1, MFAP2, WFDC1, CDH8I and LRRC2 in a sample obtained from a subject,
comparing the level of methylation of the one or more genes to a control level of methylation of the one or more genes,
determining the stage and/or type of cancer of the subject based at least in part on the difference in the level of methylation of the one or more genes in the sample compared to the control level of methylation, and
selecting a course of treatment for the subject appropriate to the stage and/or type of cancer of the subject.

67. The method of claim 66, wherein the cancer is melanoma.

68. (canceled)

69. The method of claim 66, wherein the treatment comprises chemotherapy, radiation, and/or surgical therapy.
